(19) United States
(12) Patent Application Publication
(10) Pub. No.: US 2026/0345969 A1
(43) Pub. Date: Nov. 5, 2026

(54) SYRINGE ATTACHMENTS FOR CONTROLLED INFLATION

(71) Applicant: Boston Scientific Scimed, Inc., Maple Grove, MN (US)

(72) Inventors: Andrew James Schaubhut, Bolton, MA (US); Scott E. Brechbiel, Johnston, RI (US)

(73) Assignee: Boston Scientific Scimed, Inc., Maple Grove, MN (US)

(21) Appl. No.: 19/662,543

(22) Filed: Apr. 29, 2026

Related U.S. Application Data
(60) Provisional application No. 63/798,246, filed on May 1, 2025.

(51) Int. Cl. A61M 25/10; A61B 17/12; A61B 6/00

(52) U.S. Cl. CPC A61M 25/10182; A61B 17/12136; A61B 6/481; A61B 17/1204; A61B 17/12099; A61M 2025/1052; A61M 2205/583; A61M 2205/586

(57) ABSTRACT

A syringe system for controlled inflation may include a barrel extending from a proximal end to a distal end and including a barrel flange, a plunger extending within the barrel and having a threaded region, and an adapter releasably coupled to the barrel flange. The adapter may include a body with a flexible member having an internal thread that selectively engages the plunger's threaded region. The flexible member may be movable between a first configuration where the internal thread is radially spaced from the threaded region and a second configuration where the internal thread engages the threaded region to enable controlled fluid dispensing. The adapter may include a protrusion extending distally into the barrel lumen and a lateral opening that allows assembly with the plunger.

### CROSS-REFERENCE TO RELATED APPLICATIONS

This application claims the benefit of U.S. Provisional Patent Application Serial No. 63/798,246 filed on May 1, 2025, the disclosure of which is incorporated herein by reference.

### FIELD

The disclosure pertains to medical devices, and methods for manufacturing and use of these medical devices. More particularly, the present disclosure pertains syringe attachments for controlled inflation of medical devices.

### BACKGROUND

Endoscopic retrograde cholangiopancreatography (ERCP) is a medical procedure that combines upper gastrointestinal endoscopy and x-ray imaging to diagnose and treat conditions affecting the bile ducts and the pancreatic ducts. The procedure utilizes specialized endoscopic equipment, such as a duodenoscope, which is inserted through the patient's mouth and guided through the esophagus and stomach to the duodenum. During ERCP procedures, physicians navigate the scope while maintaining control of its proximal end for various functions including irrigation, visualization, insufflation, and device management. The procedure enables the exchange of specialized devices, such as ERCP cannulas, through a biopsy port for both diagnostic and therapeutic interventions. Extraction balloons may be used to perform a balloon occlusion cholangiogram by inflating the balloon within the duct. Balloons are inflated to a range of sized depending on the size of the duct. There is an ongoing need to provide alternative inflation devices for inflating the balloon in a controlled manner, as well as alternative methods for manufacturing and using medical devices.

### SUMMARY

This disclosure provides design, material, manufacturing method, and use alternatives for medical devices and medical systems.

In an example, a syringe system for controlled inflation may include a barrel extending from a proximal end to a distal end and including a barrel flange, a plunger extending from a proximal end to a distal end disposed within the barrel where the plunger may include a plunger flange and a threaded region, and an adapter that may be releasably coupled to the barrel flange, where the adapter may include a body having a proximal side and a distal side, a flexible member extending from the body, and an internal thread on the flexible member that may be configured to selectively engage the threaded region of the plunger.

Alternatively, or additionally to any of the examples above, in another example, the flexible member may be movable between a first configuration where the internal thread may be radially spaced from the threaded region of the plunger and a second configuration where the internal thread may engage the threaded region of the plunger.

Alternatively, or additionally to any of the examples above, in another example, the flexible member may include an actuation portion that may be configured to move the flexible member between the first and second configurations.

Alternatively, or additionally to any of the examples above, in another example, the adapter may include a protrusion extending distally from the distal side of the body that may be configured to be received within a lumen of the barrel.

Alternatively, or additionally to any of the examples above, in another example, the protrusion may include a lateral opening.

Alternatively, or additionally to any of the examples above, in another example, the threaded region may include at least one of a single helix thread, a double start thread, or a triple start thread.

Alternatively, or additionally to any of the examples above, in another example, the plunger may include a forward stop extending distally from the plunger flange.

Alternatively, or additionally to any of the examples above, in another example, the plunger may include a plurality of indentations extending along a length thereof.

Alternatively, or additionally to any of the examples above, in another example, the plunger flange may include visual indicia indicating rotational direction.

Alternatively, or additionally to any of the examples above, in another example, the adapter may include an ergonomic grip pad.

Alternatively, or additionally to any of the examples above, in another example, the adapter may include a first body member and a second body member that may be releasably coupled to the first body member.

Alternatively, or additionally to any of the examples above, in another example, the first body member may include an actuatable latch that may be configured to releasably secure the second body member to the first body member.

Alternatively, or additionally to any of the examples above, in another example, the second body member may include an alignment key that may be configured to be received within a mating slot of the first body member.

Alternatively, or additionally to any of the examples above, in another example, the first body member may include a protrusion that may be configured to be received within a lumen of the barrel.

Alternatively, or additionally to any of the examples above, in another example, the second body member may include a protrusion that may be configured to be received within a lumen of the protrusion of the first body member.

In another example, a syringe system for controlled inflation may include a barrel extending from a proximal end to a distal end and including a barrel flange, a plunger movably disposed within the barrel and extending from a proximal end to a distal end where the plunger may include a plunger flange and a threaded region, and an adapter that may be releasably coupled to the barrel flange, where the adapter may include a body having a proximal side and a distal side, a flexible member extending from the body, and an internal thread on the flexible member that may be configured to selectively engage the threaded region of the plunger.

Alternatively, or additionally to any of the examples above, in another example, the flexible member may be movable between a first configuration where the internal thread may be radially spaced from the threaded region of the plunger and a second configuration where the internal thread may engage the threaded region of the plunger.

Alternatively, or additionally to any of the examples above, in another example, the flexible member may include an actuation portion that may be configured to move the flexible member between the first and second configurations.

Alternatively, or additionally to any of the examples above, in another example, the adapter may include a protrusion extending distally from the distal side of the body that may be configured to be received within a lumen of the barrel.

Alternatively, or additionally to any of the examples above, in another example, the protrusion may include a lateral opening.

Alternatively, or additionally to any of the examples above, in another example, the threaded region may include at least one of a single helix thread, a double start thread, or a triple start thread.

Alternatively, or additionally to any of the examples above, in another example, the plunger may include a forward stop extending distally from the plunger flange.

Alternatively, or additionally to any of the examples above, in another example, the plunger may include a plurality of indentations extending along a length thereof.

Alternatively, or additionally to any of the examples above, in another example, the plunger flange may include visual indicia indicating rotational direction.

Alternatively, or additionally to any of the examples above, in another example, the adapter may include an ergonomic grip pad.

In another example, a syringe adapter system may include a first body member including a recess that may be configured to receive a barrel flange of a syringe barrel, a second body member that may be releasably coupled to the first body member, where at least one of the first and second body members may include an internal thread that may be configured to engage a threaded region of a plunger.

Alternatively, or additionally to any of the examples above, in another example, the first body member may include an actuatable latch that may be configured to releasably secure the second body member to the first body member.

Alternatively, or additionally to any of the examples above, in another example, the second body member may include an alignment key that may be configured to be received within a mating slot of the first body member.

Alternatively, or additionally to any of the examples above, in another example, the first body member may include a protrusion that may be configured to be received within a lumen of the syringe barrel.

Alternatively, or additionally to any of the examples above, in another example, the second body member may include a protrusion that may be configured to be received within a lumen of the protrusion of the first body member.

In another example, a method of using a syringe system may include assembling an adapter with a barrel and plunger, filling the barrel with inflation fluid, coupling the syringe to a balloon catheter, actuating a flexible member of the adapter, and distally advancing the plunger to dispense fluid to inflate a balloon to a first diameter.

Alternatively, or additionally to any of the examples above, in another example, the method may include rotating the plunger to engage a threaded region of the plunger with an internal thread of the flexible member.

Alternatively, or additionally to any of the examples above, in another example, the method may include continuing to rotate the plunger to incrementally increase the diameter of the balloon.

Alternatively, or additionally to any of the examples above, in another example, the method may include releasing the flexible member to allow linear actuation of the plunger.

Alternatively, or additionally to any of the examples above, in another example, assembling the adapter may include laterally advancing the adapter over the plunger and distally advancing the adapter to engage a barrel flange.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 is a schematic view of an illustrative system for accessing an opening of a body lumen of a subject;

FIG. 2 is a perspective view of an illustrative syringe system that may be used to provide precise, controlled inflation of the balloon;

FIG. 3 is an exploded perspective view of the illustrative syringe system;

FIGS. 4A and 4B depict perspective views of a distal side and proximal side of the illustrative adapter of FIG. 3;

FIG. 5 depicts a proximal perspective view of the syringe system of FIG. 3 in a partially assembled configuration;

FIG. 6 depicts a cross-sectional view of the illustrative adapter of FIG. 3, taken at line 6-6 of FIG. 3;

FIG. 7 depicts a cross-sectional view of the syringe system of FIG. 2, taken at line 7-7 of FIG. 2;

FIG. 8 is a flow chart of an illustrative method of using the syringe system of FIG. 2;

FIGS. 9A and 9B depict perspective views of another illustrative adapter from distal and proximal sides;

FIGS. 10A and 10B depict perspective views of another illustrative adapter from distal and proximal sides;

FIG. 11 depicts a cross-sectional view of the adapter of FIGS. 10A and 10B;

FIGS. 12A and 12B depict perspective views of another illustrative adapter from distal and proximal sides;

FIG. 13 depicts a cross-sectional view of the adapter of FIGS. 12A and 12B;

FIGS. 14A and 14B depict perspective views of another illustrative adapter;

FIG. 15 depicts a cross-sectional view of the adapter of FIGS. 14A and 14B;

FIG. 16 depicts an exploded perspective view of the adapter of FIGS. 14A and 14B;

FIG. 17 depicts the adapter of FIGS. 14A and 14B partially disassembled from the barrel and plunger;

FIG. 18 depicts a proximal perspective view of another illustrative adapter; and

FIGS. 19A and 19B depict perspective views of another illustrative adapter that positions internal threads off-axis from the primary axis of syringe operation.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Although configurations of the present disclosure may be described with specific reference to medical devices and systems (e.g., balloon retrieval catheters for stone removal) during an endoscopic retrograde cholangiopancreatography (ERCP) procedure, it should be appreciated that such medical devices and systems may be used in a variety of medical procedures which require inflation of a balloon, or other expandable device, including, for example, balloon angioplasty/angiography procedures, gastrointestinal dilation balloons, urological balloon dilaters, endotracheal/bronchial balloon devices, neurological balloon catheters, or the like.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to effect the particular feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example configurations of the disclosure.

Endoscopic retrograde cholangiopancreatography (ERCP) may be used to diagnose and/or treat conditions of the common bile duct, including, for example, gallstones, inflammatory strictures, leaks (e.g., from trauma, surgery, etc.), and cancer. In an ERCP procedure, a physician may view, through an endoscope, the inside of the stomach and/or the duodenum. Often, dyes may be injected (e.g., via a lumen of a sphincterotome or other device) into the ducts in the biliary tree and pancreas so that the area can be seen using X-rays.

During an ERCP procedure, a number of steps are typically performed while the patient is often sedated and/or anaesthetized. For example, an endoscope or duodenoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Due to the shape of the papilla, and the angle at which the common bile and pancreatic ducts meet the wall of the duodenum, the distal end of the endoscope or duodenoscope is generally placed just past the papilla. Due to this positioning beyond the papilla, the endoscopes or duodenoscopes typically used in these procedures are usually side-viewing devices. The side-viewing feature provides imaging along the lateral aspect of the tip rather than from the end of the endoscope. Such orientation may allow a clinician to obtain an image of the medial wall of the duodenum, where the papilla of Vater is located, even though the distal tip of the viewing device is beyond the opening. Once the papilla or a target area in the duodenum is visually located, an accessory medical device, such as a retrieval or extractor balloon catheter, sphincterotome, cannula, or other accessory medical device, may be extended out a side opening or window of the endoscope or duodenoscope for facilitating access through the papilla and into a desired one of the common bile duct and the pancreatic duct.

A balloon catheter is a commonly used tool for both diagnosis and for treatment of strictures and stone obstructions. The balloon may be used to perform a balloon occlusion cholangiogram by inflating the balloon within the duct, which encourages injected contrast to be delivered into the pancreatico-biliary system, while the balloon occludes the ducts near the papillary orifice to discourage any leakage of contrast out of the system. Balloons may also be used to sweep ducts clear of sludge, debris, infectious material, stones or stone fragments, or the like. Balloons may be inflated to a range of different sizes depending on the size of the duct. Inflation of the balloon may usually be done by an assistant to the endoscopist physician performing the procedure. Communication between the assistant and the physician is key to the inflation and deflation steps. In some cases, a standard syringe with a back stop indentation and inflation lines may be used to inflate or expand the balloon to the correct size. Sometimes these lines may be hard to visualize and precisely line up the plunger of the syringe therewith. Mid-range sizes may also be difficult to control. Disclosed herein are syringes and syringe attachments that may provide controlled inflation of a balloon.

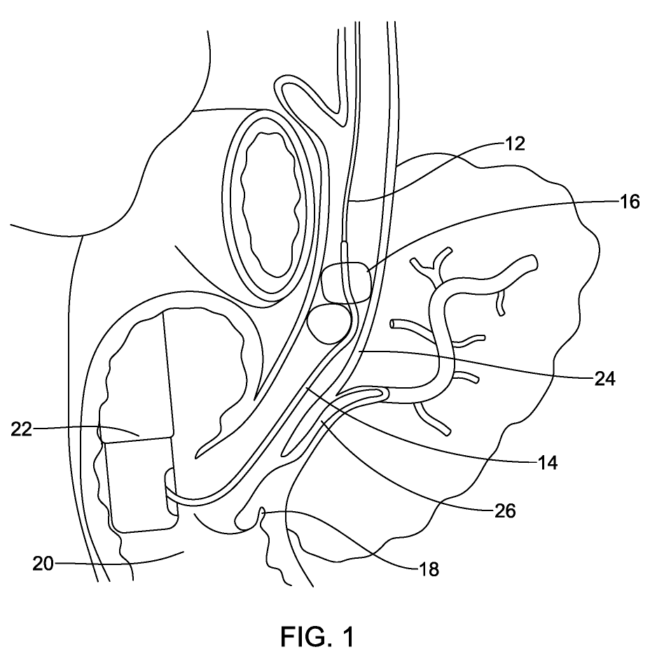

Turning to the Figures, FIG. 1 depicts a selective cannulation during an ERCP procedure, which includes a guidewire 12 and/or an accessory medical device, such as, but not limited to, a balloon catheter 14 having an expandable balloon 16 being passed towards, against, and/or through a body lumen such as the major papilla 18 (e.g., ampullary entry) near the descending duodenum 20 to access the Sphincter of Oddi Complex. During the cannulation procedure, a distal portion of a medical device 22 (e.g., an endoscope, duodenoscope, guide catheter, etc.) may be positioned within the descending duodenum 20. The guidewire 12 and the balloon catheter 14 may be advanced through a working channel (e.g., a lumen) of the medical device 22 towards the major papilla 18. Additionally, the guidewire 12 and/or the balloon catheter 14 may be advanced against and/or through the major papilla 18 to one of the common bile duct 24 and the pancreatic duct 26. Once the balloon catheter 14 is in place, the balloon 16 may be inflated to the desired diameter or desired cross-sectional dimension.

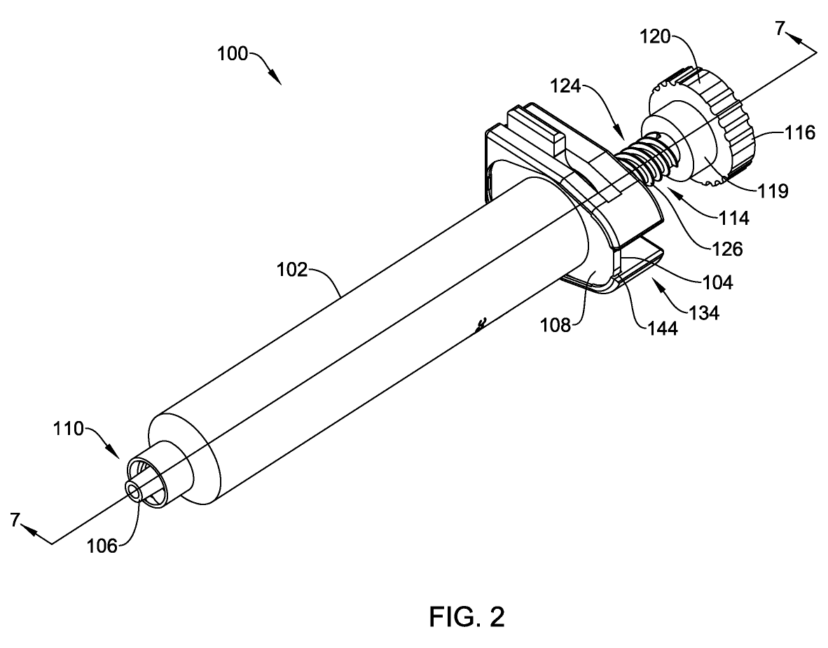

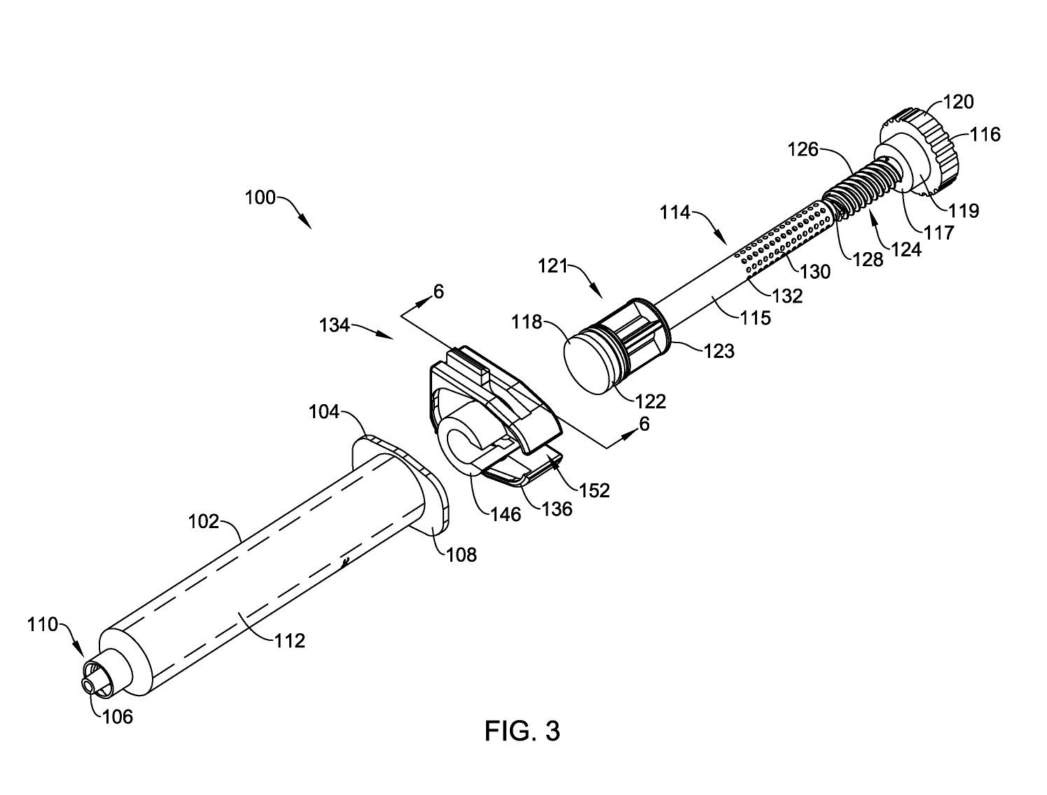

FIG. 2 is a perspective view of an illustrative syringe system 100 that may be used to provide precise, controlled inflation of the balloon 16. FIG. 3 is an exploded perspective view of the illustrative syringe system 100. Generally, the syringe system 100 may be operated to provide a mechanical stop when the balloon 16 has been inflated to a predetermined minimum diameter or volume. The syringe system 100 may then be operated to incrementally increase the diameter of the balloon 16 to a diameter between the minimum diameter and a predetermined maximum diameter or volume. It is further contemplated that incremental adjustment may be by-passed to expand or inflate the balloon 16 to the predetermined maximum diameter without incremental inflation thereof.

The syringe system 100 may include a barrel 102 extending from a proximal end 104 to a distal end 106. The proximal end 104 of the barrel 102 may include a barrel flange 108. The distal end 106 of the barrel 102 may include a Luer lock 110 configured to be releasably coupled with a proximal end (not explicitly shown) of the balloon catheter 14 to provide a leak-free or fluid tight seal therewith. A lumen 112 configured to receive a fluid therein may extend between the proximal end 104 and the distal end 106 of the barrel 102. While not explicitly shown, a cap may be removably disposed over the Luer lock 110. The cap may provide a fluid-tight seal with a distal opening of the barrel 102 to allow fluid to be stored or pre-loaded within the lumen 112, if so desired.

The syringe system 100 may further include a plunger 114 extending from a proximal end 116 to a distal end 118. The proximal end 116 of the plunger 114 may include a plunger flange 120. A forward stop 119 may extend distally from a distal surface of the plunger flange 120. Generally, the forward stop 119 may limit distal movement of the plunger 114 relative to the barrel 102. The distal end 118 of the plunger 114 may include a plunger tip 121 including a plunger seal 122 configured to form a fluid-tight seal with the luminal surface of the barrel 102. A plunger shaft 115 may extend between a distal end of the forward stop 119 and a proximal end of the plunger tip 121. In some configurations, the plunger shaft 115 may have an outer diameter or cross-sectional dimension that is less than the plunger flange 120, the forward stop 119, and/or the plunger tip 121. However, this is not required. Further, the outer diameter of the plunger shaft 115 may vary along a length thereof. The plunger 114, plunger tip 121, and plunger seal 122 may be axially and/or longitudinally slidable with respect to the barrel 102 to push materials out of and/or to aspirate materials into the lumen 112 of the barrel 102.

The syringe system 100 may further include a collar or an adapter 134 configured to be releasably secured to the barrel flange 108. Generally, the adapter 134 may be configured to selectively engage features on the plunger 114 to inflate the balloon 16 in a precise manner, as will be described in more detail herein.

In some configurations, the plunger shaft 115 may include a threaded region 124 including one or more helically extending threads 126 extending distally from a distal end of the forward stop 119. The threaded region 124 may have a length such that a distal end 128 of the threaded region 124 is configured to selectively engage a mating stop feature on the adapter 134 when the plunger 114 has been distally advanced by a length configured to fill the balloon 16 to a minimum volume or a smallest size. In some cases, the one or more helically extending threads 126 may be a single helix. In other configurations, the one or more helically extending threads 126 may be a double start thread having two parallel helixes. In yet another configuration, the one or more helically extending threads 126 may be a triple start thread having three parallel helixes. It is contemplated that a multi-start thread design (e.g., two or more starts) may enable faster thread engagement with a mating feature on the adapter 134. In some configurations, the threaded region 124 may extend along an entire length of the plunger shaft 115. However, this is not required.

The plunger shaft 115 may optionally further include one or more or a plurality of recesses, through holes, or indentations 130. The indentations 130 may extend distally from the distal end 128 of the threaded region 124 to a location 132 proximal to the distal end 118 of the plunger 114. The indentations 130 may improve the gripability of the plunger for one handed use while still providing a smooth insertion through the adapter 134. In some cases, the indentations 130 may extend to a proximal end of the plunger tip 121. However, this is not required. The indentations 130 may extend along any length of the plunger shaft 115, as desired. The region of the plunger shaft 115 free from the threaded region 124 may have a diameter or cross-sectional dimension that is less than the diameter or cross-sectional dimension of the threaded region 124. In some configurations, the threaded region 124 may extend along an entirety of the length of plunger shaft 115 and the indentations 130 may be omitted.

In some configurations, the plunger 114 may be formed as a single monolithic structure. However, this is not required. In other configurations, the plunger 114 may be formed from two or more components that are subsequently secured to one another. It is contemplated that snap fits, threaded engagements, or other mechanical securing mechanisms may be used to secure two or more components. In some cases, the plunger flange 120 and forward stop 119 may be formed as a first component while plunger shaft 115 and the plunger tip 121 may be formed as a second component. In such an example, the proximal end of the plunger shaft 115 may be coupled with the distal end of the forward stop 119. In another example, the plunger flange 120, forward stop 119, and threaded region 124 of the plunger shaft 115 may be formed as a first component while the region of the plunger shaft 115 free from the threads 126 and the plunger tip 121 may be formed as a second component. In such an example, the distal end 128 of the threaded region 124 may be coupled with the proximal end region of the plunger shaft 115 free from the threads 126. In yet another example, the plunger flange 120, forward stop 119, and the plunger shaft 115 may be formed as a first component while the plunger tip 121 may be formed as a second component. In such an example, the distal end of the plunger shaft 115 may be coupled with the proximal end of the plunger tip 121. It is contemplated that the plunger 114 may be formed as three components using any of the connection locations described herein. These are just some examples of some configurations of a multi-component plunger 114 and are not intended to limit the plunger 114 to specific assembly configurations.

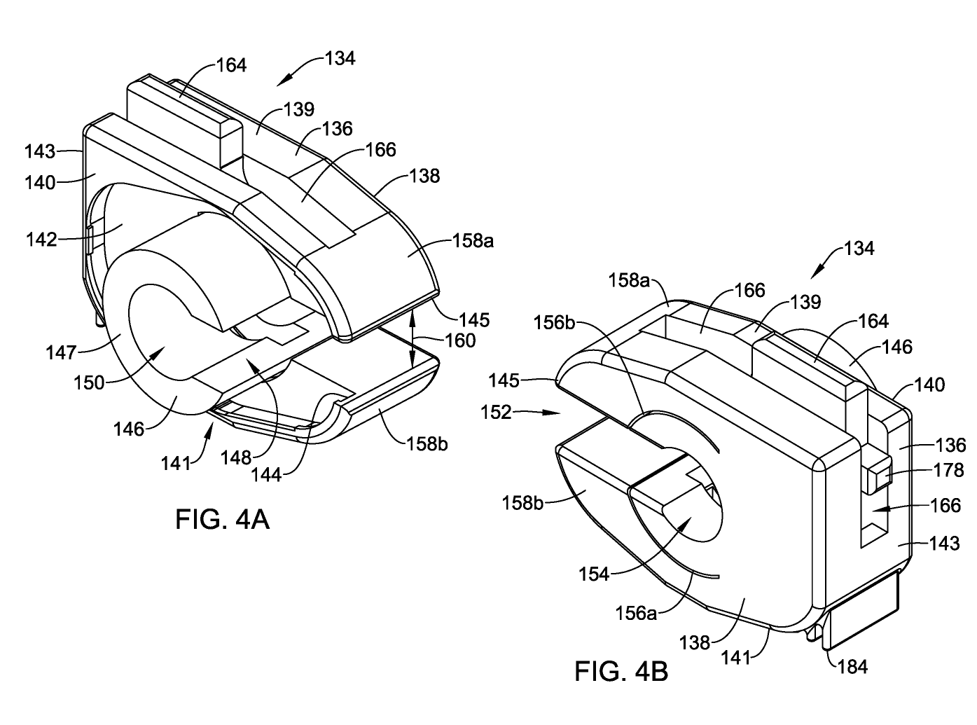

FIG. 4A is perspective view of a distal side of the adapter 134 and FIG. 4B is a perspective view of a proximal side of the adapter 134. The adapter 134 may be formed as a single monolithic structure. However, this is not required. In some examples, the adapter 134 may be formed from two or more pieces or components that are subsequently coupled together. In an example configuration, the adapter 134 may be formed from one or more molded plastic components, such as, but not limited to, acrylonitrile butadiene styrene (ABS). However, a variety of different manufacturing techniques and/or materials may be used to form the adapter 134.

The adapter 134 includes a housing or body 136 including a proximal side 138, a distal side 140, and at least a top side 139 and a second opposing bottom side 141. The top and bottom sides 139, 141 may each extend from or between the proximal side 138 to the distal side 140. The body 136 may further include a first lateral side 143 and an opposing second lateral side 145. The use of the terms “proximal”, “distal”, “first”, “second”, “top”, and “bottom” are not intended to limit the adapter 134 to a particular orientation, but rather facilitate discussion of relative orientation.

The distal side 140 may include a proximally extending recess 142 configured to receive the barrel flange 108 therein. A groove 144 may extend at least partially around a perimeter of the recess 142. In some cases, the groove 144 may extend around an entirety of the perimeter of the recess 142. However, this is not required. The perimeter of the barrel flange 108 may form a snap fit with the groove 144 to releasably secure the adapter 134 to the barrel flange 108, as shown in FIG. 2. The adapter 134 may have a cross-sectional shape that is similar to the barrel flange 108 such that the barrel flange 108 can be removably secured within the recess 142.

A generally C-shaped protrusion 146 may extend distally from the recess 142, and in some cases, distally beyond the distal side 140 of the body 136. In some cases, the protrusion 146 may be generally tubular with a portion of the sidewall removed along a length thereof to define a lateral opening 148. The lateral opening 148 may be oriented towards the second lateral side 145. The protrusion 146 may be configured to be received within the lumen 112 of the barrel 102 such that a distal end (e.g., a distal surface 147) of the protrusion 146 is disposed within the lumen 112. For example, an outer diameter of the protrusion 146 may be less than a luminal diameter of the lumen 112. In some configurations, the outer diameter of the protrusion 146 may be approximately the same as or greater than the luminal diameter of the lumen 112 to provide a friction fit between the outer surface of the protrusion 146 and the luminal surface of the lumen 112.

The lateral opening 148 of the protrusion 146 may extend along an axial length (e.g., proximal to distal direction) thereof. The lateral opening 148 may allow the side wall of the protrusion 146 to flex as needed. For example, a lumen 150 may extend along the axial length of the protrusion 146 and may be in communication with or connected to the lateral opening 148 of the protrusion 146. A diameter of the lumen 150 may expand to allow the adapter 134 to be positioned over the plunger 114 in a lateral direction and compressed when inserted into the lumen 112 of the barrel 102. These are just some examples.

The body 136 of the adapter 134 may include a lateral opening 152 opening toward the second lateral side 145 and aligned with the lateral opening 148 of the protrusion 146. The lateral opening 152 may define a first portion or arm 158a adjacent the top side 139 and a second arm or portion 158b adjacent the bottom side 141 which allow the body 136 of the adapter 134 to flex as needed. For example, the arms 158a, 158b may move away from one another to allow the adapter 134 to be laterally assembled with the plunger shaft 115. An aperture or lumen 154 may extend distally from the proximal side 138 of the body 136. The lumen 154 may extend co-axially with and connect to the lumen 150 of the protrusion 146 to form a continuous lumen. Further, the lumen 154 may be in communication with or connected to the lateral opening 152. In some cases, one or more slots or reliefs 156a, 156b may be radially spaced from the lumen 154 and extend partially around the perimeter thereof. The one or more reliefs 156a, 156b may extend distally from the proximal side 138 of the body 136 to the recess 142. It is contemplated that the reliefs 156a, 156b may be slits or slots configured to allow the diameter of the lumen 154 to temporarily expand to allow the adapter 134 to be positioned over the plunger 114 in a lateral direction. For example, the lateral opening 152 may have a height 160 that is less than a diameter of the plunger shaft 115.

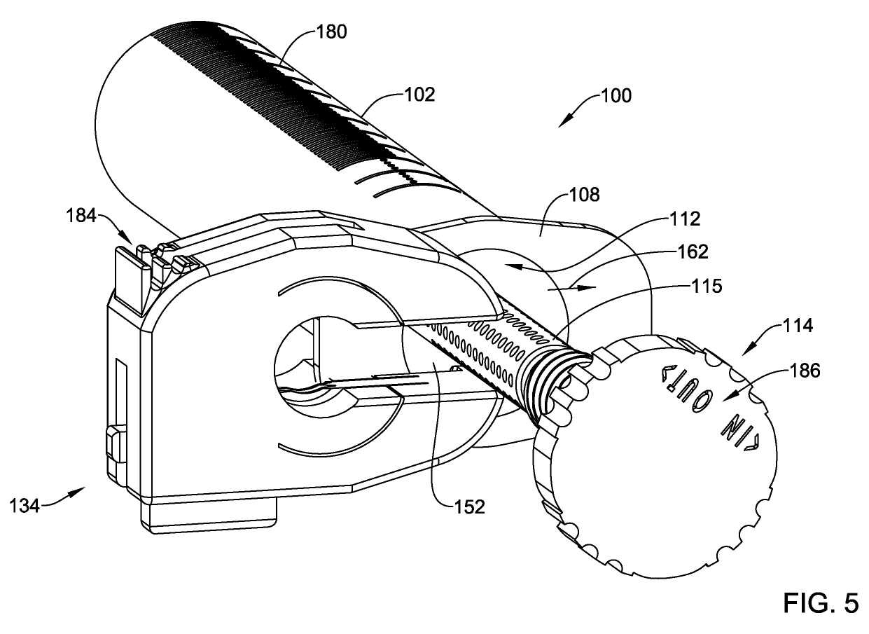

Referring briefly to FIG. 5, which illustrates a proximal perspective view of the illustrative syringe system 100 in a partially assembled configuration, to assemble the syringe system 100, the plunger tip 121 may be inserted into the lumen 112 of the barrel 102. Next, the lateral opening 152 of the adapter 134 may be aligned with the plunger shaft 115 (e.g., between the forward stop 119 and the plunger tip 121). In some cases, the lateral opening 152 may be aligned with a region of the plunger shaft 115 free from threads 126. However, this is not required. The adapter 134 may then be laterally advanced over the plunger shaft 115, as shown at arrow 162, to position the plunger shaft 115 within the lumen 154 of the body 136 and the lumen 150 of the protrusion 146. The arms 158a, 158b may flex away from one another to allow the adapter 134 to be laterally advanced and return to their original unbiased configurations when the plunger shaft 115 is disposed within the lumens 150, 154. Next, the adapter 134 may be distally advanced along the plunger shaft 115 until the barrel flange 108 is disposed with the recess 142 and a perimeter of the barrel flange 108 forms a snap fit or friction fit with the groove 144 of the adapter 134. The diameter of the lumens 150, 154 may be greater than a diameter of the plunger shaft 115 to allow the plunger 114 to move freely relative to the adapter 134 and/or the barrel 102 in the absence of an applied force to the adapter 134. In some configurations, the diameter of the lumens 150, 154 may be greater than the diameter of the plunger shaft 115 free from the threads 126 and approximately equal to and less than the diameter of the plunger shaft 115 at the threaded region 124. For example, the plunger 114 may be distally advanced in an axial manner within the lumens 150, 154 until the distal end 128 of the threaded region contacts a portion of the adapter 134 at which point, rotation of the plunger 114 may be required to further move the plunger 114. However, this is not required.

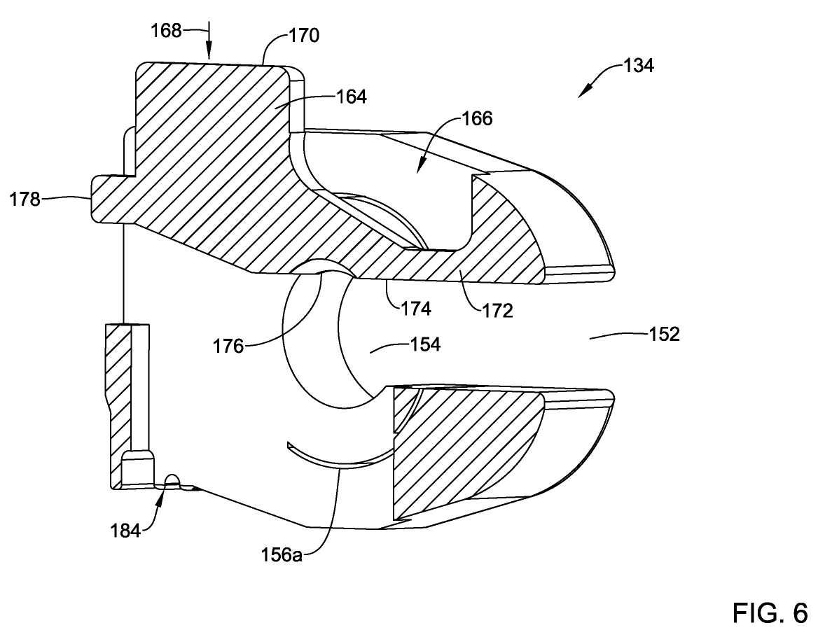

Returning to FIGS. 4A and 4B, the adapter 134 may further include a compliant hinge or flexible member 164 extending from the top side 139 of the body 136. Referring additionally to FIG. 6, which is a cross-sectional view of the adapter 134 taken at line 6-6 of FIG. 3, the flexible member 164 may be movably positioned within a slot 166 formed in the body 136 of the adapter 134. The slot 166 may extend from the top side 139 of the body 136 towards the bottom side 141 thereof. In some cases, the slot 166 may extend from the top side 139 of the bottom side 141 of the body 136. Further, at least a portion of the slot 166 may extend to the first lateral side 143 of the body 136. A radially inward surface 174 of the flexible member 164 may include an internal thread 176 or more than one internal thread configured to selectively engage the external threads 126 of the plunger 114. The one or more internal threads 176 may be aligned with the lumen 154.

The flexible member 164 may be movably coupled to or formed as a deflectable section of a single monolithic structure with the body 136. For example, a region 172 of the flexible member 164 having a reduced thickness extends from the body 136. The flexible member 164 may be configured to be biased radially inwards to selectively engage the internal thread 176 with the threaded region 124 of the plunger shaft 115. In an unbiased configuration, the internal thread 176 of the flexible member 164 may be radially spaced from an outer surface of the plunger shaft 115 such that the plunger shaft 115 (e.g., between the distal end 117 of the forward stop 119 and the proximal end 123 of the plunger tip 121) is axially displaceable within the lumens 150, 154 of the adapter 134 and the lumen 112 of the barrel 102 using linear movement of the plunger 114. The user may exert a radially inwards biasing force 168 on an actuation portion 170 of the flexible member 164 to move the internal thread 176 radially inwards. The actuation portion 170 may extend above the top 139 of the body 136 to allow the user to locate and actuate the flexible member 164 without visual verification. Upon exertion of the applied force 168, the flexible member 164 may bend or deflect at the reduced thickness region 172 to move radially inwards. In the absence of the applied force 168, the flexible member 164 may return to its original unbiased configuration where the internal thread 176 of the flexible member 164 may be radially spaced from an outer surface of the plunger shaft 115. The flexible member 164 may further include an optional tab 178 extending laterally beyond the first lateral side 143 of the body 136. The tab 178 may allow the user to move the flexible member 164 radially outwards in the event the flexible member 164 does not return to its original unbiased configurations when the radially inward force 168 is released. In some cases, the tab 178 may be omitted. The body 136 of the adapter 134 may further include an ergonomic grip pad 184 positioned along the bottom side 141 thereof. The ergonomic grip pad 184 may be positioned adjacent to the first lateral side 143 of the body 136 opposite from the actuation portion 170. The grip pad 184 may include a plurality of grooves and ridges or other texturing to improve the gripability of the adapter 134 while the flexible member 164 is actuated. It is further contemplated that the actuation portion 170 and/or other regions of the flexible member 164 may include a plurality of grooves and ridges or other texturing to improve the gripability thereof.

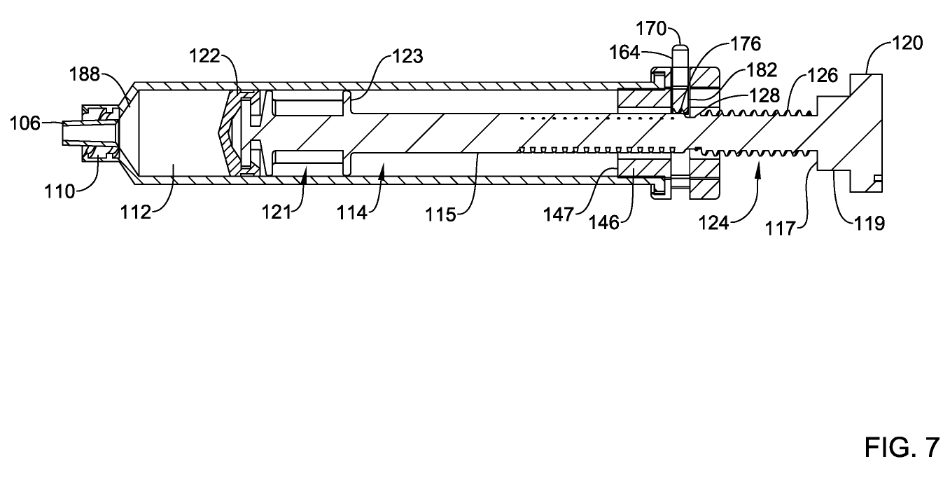

Referring additionally to FIG. 7, which is a cross-sectional view of the syringe system 100 taken at line 7-7 of FIG. 2, when the flexible member 164 is biased radially inwards, the diameter of the lumen 154 at a proximal surface 182 of the flexible member 164 (e.g., proximal to the internal thread 176) may be less than the outermost diameter of the threaded region 124. Thus, when the plunger 114 is distally advanced with the flexible member 164 biased radially inwards, the distal end 128 of the threaded region 124 may mechanically engage the flexible member 164 proximal to the internal thread 176. The distal end 128 of the threaded region 124 may be located to allow the user to precisely inject or dispense a predetermined amount of fluid into the inflatable balloon 16 without visual verification. For example, the user may inject the fluid from the syringe system 100 into the balloon 16 without using volume lines 180 (see, for example, FIG. 5) on an outer surface of the barrel 102 to determine how much fluid has been injected into the balloon 16. Once the distal end 128 of the threaded region 124 has engaged the proximal surface 182 of the flexible member 164, the user may rotate the plunger 114 in a first direction (e.g., clockwise) to engage the distalmost thread 126 with the internal thread 176. Continued rotation of the plunger 114 in the first direction may distally advance the plunger 114 within the lumen 112 of the barrel 102 to dispense additional fluid from the lumen 112 of the barrel 102 to the balloon 16 in a controlled and/or precise manner. In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102.

In some configurations, the plunger flange 120 may be sized and/or shaped to allow the user to easily count rotations of the plunger 114. For example, in some examples, the plunger flange 120 may have generally square or generally rectangular cross-sectional shape which may allow the user to precisely rotate the plunger 114 in quarter turn increments. In other examples, the plunger flange 120 may take other polygonal or non-polygonal (e.g., circular, oval, or the like) cross-sectional shapes. Alternatively, or additionally, the plunger flange 120 may include visual markings or indexing to allow a user to count rotations. In yet another example, the plunger flange 120 may include physical indicators such as a protrusion or recess in an outer perimeter thereof to allow a user to count rotations of the plunger 114. In some cases, a plunger flange 120 having a generally circular cross-sectional shape may have a flat section for tactile orientation. The plunger flange 120 may further include features such as, but not limited to, ridges and valleys extending around the perimeter thereof to increase gripability thereof. In some configurations, the plunger flange 120 may include visual indicia 186 (see, for example, FIG. 5) providing the user with directional indictors indicating which direction to rotate the plunger 114 for inflation and deflation.

The user may rotate the plunger 114 until the distal end 117 of the forward stop 119 contacts the proximal side 138 of the body 136 of the adapter 134. However, the user may stop rotation of the plunger 114 at any time before the distal end 117 of the forward stop 119 contacts the proximal side 138 of the body 136 if the desired diameter of the balloon 16 has been reached. The length of the plunger shaft 115 may be selected such that when the distal end 117 of the forward stop 119 contacts the proximal side 138 of the adapter 134, the balloon 16 has been inflated to a largest size (e.g., largest diameter) or a maximum allowable volume has been dispensed. In other examples, the user may rotate the plunger 114 until the plunger seal 122 contacts a tapered distal end 188 of the lumen 112. In such an instance, the distal end 117 of the forward stop may be spaced from the proximal side 138 of the body 136 when a maximum volume of fluid has been dispensed.

It is contemplated that if the user does not wish to incrementally inflate the balloon 16 either after the minimum volume has been dispensed (to achieve the smallest balloon size) or prior to dispensing any fluid, the flexible member 164 may be released to allow the user to inflate the balloon to the largest size without requiring rotation of the plunger 114. Said differently, the syringe system 100 may be operated in a manner consistent with a typical syringe, or the flexible member 164 may be depressed to allow controlled volumes to be dispensed.

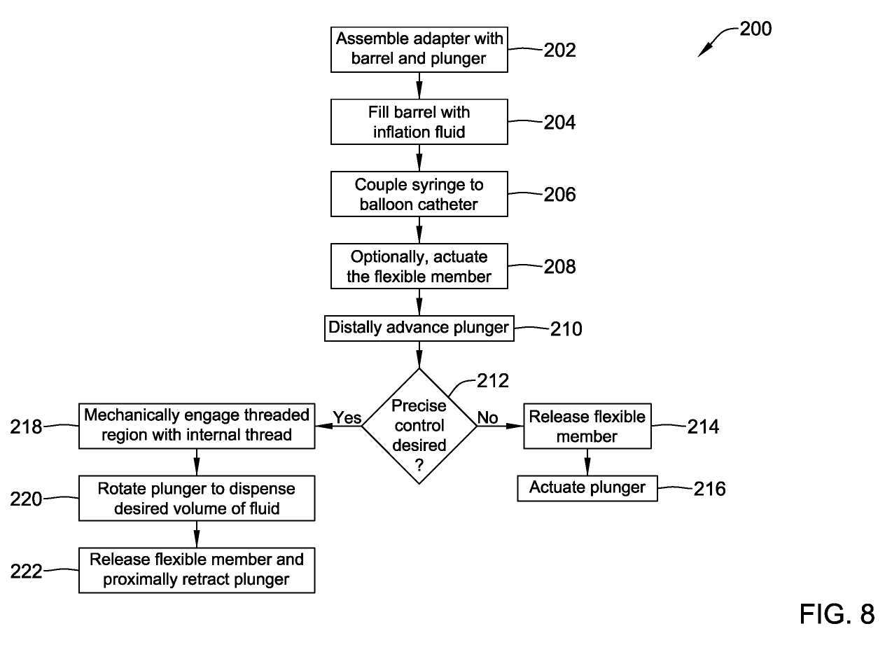

FIG. 8 is a flow chart of an illustrative method 200 of using the syringe system 100. While certain steps are shown and described as a sequence, in other embodiments fewer steps are contemplated and the order by which steps are performed can be different than what is described. To begin, the adapter 134 may be assembled with the barrel 102 and plunger 114, as shown at block 202. As described herein, to assemble the adapter 134 with the barrel 102 and plunger 114, the plunger tip 121 may be inserted into the lumen 112 of the barrel 102. However, it is not required that the plunger tip 121 be inserted into the lumen 112 as a first step. Next, the lateral opening 152 of the adapter 134 may be aligned with the plunger shaft 115 (e.g., between the forward stop 119 and the plunger tip 121). The adapter 134 may then be laterally advanced over the plunger shaft 115, as shown at arrow 162, to position the plunger shaft 115 within the lumen 154 of the body 136 and the lumen 150 of the protrusion 146. The arms 158a, 158b may flex away from one another to allow the adapter 134 to be laterally advanced. Next, the adapter 134 may be distally advanced along the plunger shaft 115 until the barrel flange 108 is disposed with the recess 142 and a perimeter of the barrel flange 108 forms a snap or friction fit with the groove 144 of the adapter 134.

Next, the barrel 102 may be filled with inflation fluid, as shown at block 204. To fill the lumen 112 of the barrel 102, the user may move the plunger 114 to a distalmost configuration. In the distalmost configuration, the distal end 117 of the forward stop 119 may mechanically engage the proximal side 138 of the adapter 134 or the plunger seal 122 may contact a distal end 188 of the lumen 112. The plunger 114 may be axially displaced (e.g., distally advanced) with the flexible member 164 in an unbiased configuration such that the internal thread 176 is radially spaced from an outer surface of the plunger shaft 115. This may allow the plunger 114 to be axially displaced in a linear manner without requiring rotation of the plunger 114. Next, the distal end 106 of the barrel 102 may be placed in fluid communication with the inflation fluid and the plunger 114 proximally retracted to pull the inflation fluid into the lumen 112. The plunger 114 may be proximally retracted with the flexible member 164 in an unbiased configuration such that the internal thread 176 is radially spaced from an outer surface of the plunger shaft 115. This may allow the plunger 114 to be axially displaced without requiring rotation of the plunger 114. The plunger 114 may be proximally retracted until a proximal end 123 (e.g., a proximal surface of the proximal end 123) of the plunger tip 121 mechanically engages or contacts a distal surface 147 of the protrusion 146 of the adapter 134. It is contemplated that the length of the protrusion 146 may be selected to allow a predetermined volume of inflation fluid to be pulled into the lumen 112 of the barrel 102. The predetermined volume may correspond to the volume of inflation fluid required to inflate or expand the balloon 16 to the largest size or a maximum diameter. Further, the engagement between the proximal end 123 (e.g., the proximal surface of the proximal end 123) of the plunger tip 121 and the distal surface 147 of the protrusion 146 of the adapter 134 may prevent accidental disassembly of the plunger 114 from the barrel 102.

Prior to or at the desired time of inflation, the Luer lock 110 of the syringe system 100 may be coupled with a proximal end of the balloon catheter 14 to fluidly couple the syringe system 100 with the balloon 16, as shown at block 206. When inflation of the balloon 16 is desired, the user may optionally depress the actuation portion 170 of the flexible member 164 to move the internal thread 176 radially inwards, as shown at block 208. The user may then distally advance the plunger 114 within the barrel 102, as shown at block 210. When the actuation portion 170 is depressed, distal advancement of the plunger 114 may continue until the distal end 128 of the threaded region 124 contacts the proximal surface 182 of the flexible member 164 to prevent further distal movement of the plunger 114. This mechanical stop may indicate to the user that the balloon 16 has been inflated to a smallest predetermined size.

The user may then determine if it is desired to continue to expand the balloon 16 in an incremental or controlled manner, as shown at block 212. If the user does not desire to further inflate the balloon 16 or if precise control is not desired, the user may release the actuation portion 170 to allow the flexible member 164 to return to an original unbiased configuration in which the internal thread 176 is radially spaced from an outer surface of the plunger shaft 115, as shown at block 214. The user may then distally advance the plunger 114 (without rotation) to further inflate the balloon 16 or proximally retract the plunger 114 (without rotation) to deflate the balloon 16, as shown at block 216.

If the user desires to expand the balloon 16 in an incremental or controlled manner, the user may rotate the plunger 114 to mechanically engage the threaded region 124 (e.g., as distalmost thread 126 thereof) with the internal thread 176 of the flexible member 164, as shown at block 218. The user may then rotate the plunger 114 to dispense the desired volume of fluid or until the desired balloon size is achieved, as shown at block 220. When deflation of the balloon 16 is desired, the user may release the actuation portion 170 of the flexible member 164 to allow the flexible member 164 to return to an original unbiased configuration in which the internal thread 176 is radially spaced from an outer surface of the plunger shaft 115 and proximally retract the plunger 114 in a linear manner, as shown at block 222.

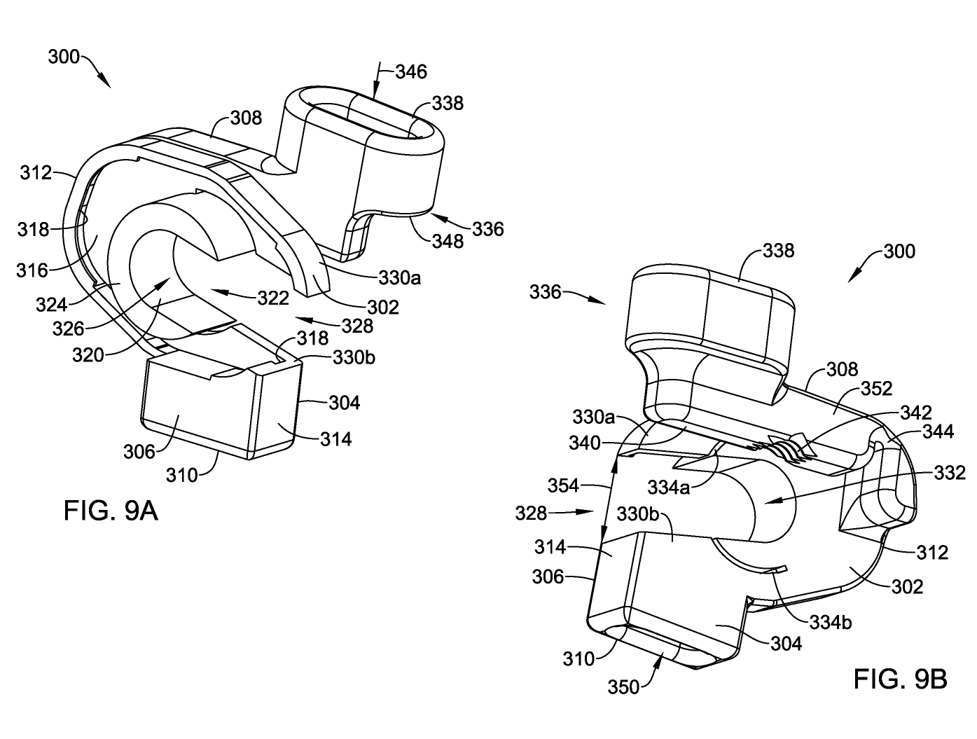

FIG. 9A is perspective view of a distal side of another illustrative adapter 300 and FIG. 9B is a perspective view of a proximal side of the adapter 300. The adapter 300 may be used with the barrel 102 and plunger 114 in place of the adapter 134 described herein. The adapter 300 may be formed as a single monolithic structure. However, this is not required. In some examples, the adapter 300 may be formed from two or more pieces or components that are subsequently coupled together. In an example configuration, the adapter 300 may be formed from one or more molded plastic components, such as, but not limited to acrylonitrile butadiene styrene (ABS). However, a variety of different manufacturing techniques and/or materials may be used to form the adapter 300.

The adapter 300 includes a housing or body 302 including a proximal side 304, a distal side 306, and at least a top side 308 and a second opposing bottom side 310. The top and bottom sides 308, 310 may each extend from or between the proximal side 304 to the distal side 306. The body 302 may further include a first lateral side 312 and an opposing second lateral side 314. The use of the terms “proximal”, “distal”, “first”, “second”, “top”, and “bottom” are not intended to limit the adapter 300 to a particular orientation, but rather facilitate discussion of relative orientation.

The distal side 306 may include a proximally extending recess 316 configured to receive the barrel flange 108 therein. A groove 318 may extend at least partially around a perimeter of the recess 316. In some cases, the groove 318 may extend around an entirety of the perimeter of the recess 316. However, this is not required. The perimeter of the barrel flange 108 may form a snap fit with the groove 318 to releasably secure the adapter 300 to the barrel flange 108.

A generally C-shaped protrusion 320 may extend distally from the recess 316, and in some cases, distally beyond the distal side 306 of the body 302. In some cases, the protrusion 320 may be generally tubular with a portion of the sidewall removed along a length thereof to define a lateral opening 322. The lateral opening may be oriented towards the second lateral side 314. The protrusion 320 may be configured to be received within the lumen 112 of the barrel 102 such that a distal end 324 of the protrusion 320 is disposed within the lumen 112. For example, an outer diameter of the protrusion 320 may be less than a luminal diameter of the lumen 112. In some configurations, the outer diameter of the protrusion 320 may be approximately the same as or greater than the luminal diameter of the lumen 112 to provide a friction fit between the outer surface of the protrusion 320 and the luminal surface of the lumen 112.

The lateral opening 322 of the protrusion 320 may extend along an axial length (e.g., proximal to distal direction) thereof. The lateral opening 322 may allow the side wall of the protrusion 320 to flex as needed. For example, a lumen 326 may extend along the axial length of the protrusion 320 and may be in communication with or connected to the lateral opening 322 of the protrusion 320. A diameter of the lumen 326 may expand to allow the adapter 300 to be positioned over the plunger 114 in a lateral direction and compressed when inserted into the lumen 112 of the barrel 102. These are just some examples.

The body 302 of the adapter 300 may include a lateral opening 328 opening toward the second lateral side 314 and aligned with the lateral opening 322 of the protrusion 320. The lateral opening 328 may define a first portion or arm 330a adjacent the top side 308 and a second arm or portion 330b adjacent the bottom side 310 which allow the body 302 of the adapter 300 to flex as needed. For example, the arms 330a, 330b may move away from one another to allow the adapter 300 to be laterally assembled with the plunger shaft 115. An aperture or lumen 332 may extend distally from the proximal side 304 of the body 302. The lumen 332 may extend co-axially with and connect to the lumen 326 of the protrusion 320 to form a continuous lumen. Further, the lumen 332 may be in communication with or connected to the lateral opening 328. In some cases, one or more slots or reliefs 334a, 334b may be radially spaced from the lumen 332 and extend partially around the perimeter thereof. The one or more reliefs 334a, 334b may extend distally from the proximal side 304 of the body 302 to the recess 316. It is contemplated that the reliefs 334a, 334b may be slits or slots configured to allow the diameter of the lumen 332 to temporarily expand to allow the adapter 300 to be positioned over the plunger 114 in a lateral direction in a similar manner to adapter 134. For example, the lateral opening 328 may have a height 354 that is less than a diameter of the plunger shaft 115.

The adapter 300 may further include a compliant hinge or flexible member 336 extending proximally from the proximal side 304 of the body 302. The flexible member 336 may include an actuation portion 338 extending above the top side 308 of the body 302 to allow the user to easily locate the actuation portion 338. A radially inward surface 340 of the flexible member 336 may include one or more internal threads 342 configured to selectively engage the external threads 126 of the plunger 114. The one or more internal threads 342 may be aligned with the lumen 332.

The flexible member 336 may be movably coupled to or formed as a deflectable section of a single monolithic structure with the body 302. For example, a region 344 of the flexible member 336 having a reduced thickness extends from the body 302. The flexible member 336 may be configured to be biased radially inwards (e.g., towards the lumen 332) to selectively engage the one or more internal threads 342 with the threaded region 124 of the plunger shaft 115. In an unbiased configuration, the one or more internal threads 342 of the flexible member 336 may be radially spaced from an outer surface of the plunger shaft 115 such that the plunger shaft 115 (e.g., between the distal end 117 of the forward stop 119 and the proximal end 123 of the plunger tip 121) is axially displaceable within the lumens 326, 332 of the adapter 300 and the lumen 112 of the barrel 102 using linear movement of the plunger 114. The user may exert a radially inwards biasing force 346 on the actuation portion 338 of the flexible member 336 to move the one or more internal threads 342 radially inwards towards the lumen 332. The actuation portion 338 may extend above the top 308 of the body 302 to allow the user to locate and actuate the flexible member 336 without visual verification. Upon exertion of the applied force 346, the flexible member 336 may bend or deflect at the reduced thickness region 344 to move radially inwards. In the absence of the applied force 346, the flexible member 336 may return to its original unbiased configuration where the one or more internal threads 342 of the flexible member 336 may be radially spaced from an outer surface of the plunger shaft 115. The flexible member 336 may further include a tab or ledge 348 formed in the flexible member 336. The ledge 348 may allow the user to move the flexible member 336 radially outwards in the event the flexible member 336 does not return to its original unbiased configurations when the radially inward force 346 is released. The body 302 of the adapter 300 may further include an ergonomic grip pad 350 positioned along the bottom side 310 thereof. The ergonomic grip pad 350 may be positioned adjacent to the second lateral side 314 of the body 302 opposite from the actuation portion 338. The grip pad 350 may include a recess, a plurality of grooves and ridges, or other texturing to improve the gripability of the adapter 300 while the flexible member 336 is actuated. It is further contemplated that the actuation portion 338 and/or other regions of the flexible member 336 may include a plurality of grooves and ridges or other texturing to improve the gripability thereof.

When the flexible member 336 is biased radially inwards, the diameter of the lumen 332 at a proximal surface 352 of the flexible member 336 (e.g., proximal to the internal thread 342) may be less than the outermost diameter of the threaded region 124. Thus, when the plunger 114 is distally advanced with the flexible member 336 biased radially inwards, the distal end 128 of the threaded region 124 may mechanically engage the flexible member 336 proximal to the internal thread. The distal end 128 of the threaded region 124 may be located to allow the user to precisely inject or dispense a predetermined amount of fluid into the inflatable balloon 16 without visual verification. For example, the user may inject the fluid from the syringe system 100 into the balloon 16 without using volume lines on an outer surface of the barrel 102 to determine how much fluid has been injected into the balloon 16. Once the distal end 128 of the threaded region 124 has engaged the proximal surface 352 of the flexible member 336, the user may rotate the plunger 114 in a first direction (e.g., clockwise) to engage the distalmost thread 126 with the one or more internal threads 342. Continued rotation of the plunger 114 in the first direction may distally advance the plunger 114 within the lumen 112 of the barrel 102 to dispense additional fluid from the lumen 112 of the barrel 102 to the balloon 16 in a controlled and/or precise manner. In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102.

The user may rotate the plunger 114 until the distal end 117 of the forward stop 119 contacts the proximal side 304 of the body 302 of the adapter 300. However, the user may stop rotation of the plunger 114 at any time before the distal end 117 of the forward stop 119 contacts the proximal side 304 of the body 302 if the desired diameter of the balloon 16 has been reached. The length of the plunger shaft 115 may be selected such that when the distal end 117 of the forward stop 119 contacts the proximal side 304 of the adapter 300, the balloon 16 has been inflated to a largest size (e.g., largest diameter) or a maximum allowable volume has been dispensed. In other examples, the user may rotate the plunger 114 until the plunger seal 122 contacts a tapered distal end 188 of the lumen 112. In such an instance, the distal end 117 of the forward stop may be spaced from the proximal side 138 of the body 136 when a maximum volume of fluid has been dispensed.

It is contemplated that if the user does not wish to incrementally inflate the balloon 16 either after the minimum volume has been dispensed (to achieve the smallest balloon size) or prior to dispensing any fluid, the flexible member 336 may be released to allow the user to inflate the balloon to the largest size without requiring rotation of the plunger 114. Said differently, the syringe system 100 may be operated in a manner consistent with a typical syringe, or the flexible member 336 may be depressed to allow controlled volumes to be dispensed. It is contemplated that the adapter 300 may be used with the barrel 102 and plunger 114 in a similar manner to that described with respect to adapter 134.

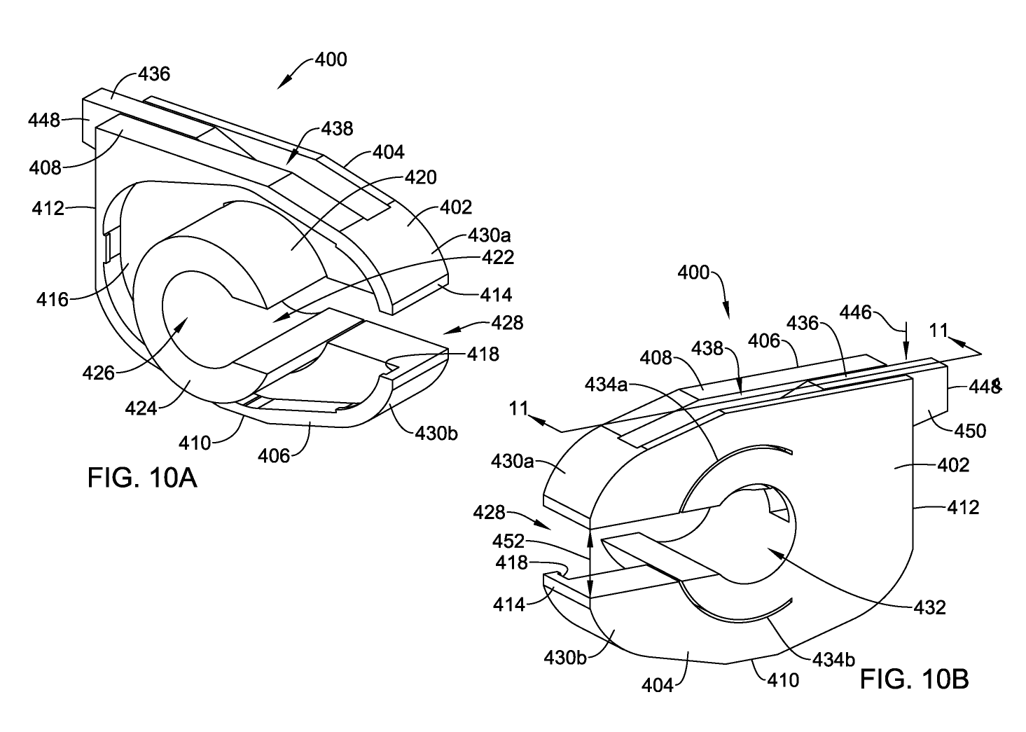

FIG. 10A is perspective view of a distal side of another illustrative adapter 400 and FIG. 10B is a perspective view of a proximal side of the adapter 400. The adapter 400 may be used with the barrel 102 and plunger 114 in place of the adapter 134 described herein. The adapter 400 may be formed as a single monolithic structure. However, this is not required. In some examples, the adapter 400 may be formed from two or more pieces or components that are subsequently coupled together. In an example configuration, the adapter 400 may be formed from one or more molded plastic components, such as, but not limited to acrylonitrile butadiene styrene (ABS). However, a variety of different manufacturing techniques and/or materials may be used to form the adapter 400.

The adapter 400 includes a housing or body 402 including a proximal side 404, a distal side 406, and at least a top side 408 and a second opposing bottom side 410. The top and bottom sides 408, 410 may each extend from or between the proximal side 404 to the distal side 406. The body 402 may further include a first lateral side 412 and an opposing second lateral side 414. The use of the terms “proximal”, “distal”, “first”, “second”, “top”, and “bottom” are not intended to limit the adapter 400 to a particular orientation, but rather facilitate discussion of relative orientation.

The distal side 406 may include a recess 416 configured to receive the barrel flange 108 therein. A groove 418 may extend at least partially around a perimeter of the recess 416. In some cases, the groove 418 may extend around an entirety of the perimeter of the recess 416. However, this is not required. The perimeter of the barrel flange 108 may form a snap fit with the groove 418 to releasably secure the adapter 400 to the barrel flange 108.

A generally C-shaped protrusion 420 may extend distally from the recess 416, and in some cases, distally beyond the distal side 406 of the body 402. In some cases, the protrusion 420 may be generally tubular with a portion of the sidewall removed along a length thereof to define a lateral opening 422. The lateral opening may be oriented towards the second lateral side 414. The protrusion 420 may be configured to be received within the lumen 112 of the barrel 102 such that a distal end 424 of the protrusion 420 is disposed within the lumen 112. For example, an outer diameter of the protrusion 420 may be less than a luminal diameter of the lumen 112. In some configurations, the outer diameter of the protrusion 420 may be approximately the same as or greater than the luminal diameter of the lumen 112 to provide a friction fit between the outer surface of the protrusion 420 and the luminal surface of the lumen 112.

The lateral opening 422 of the protrusion 420 may extend along an axial length (e.g., proximal to distal direction) thereof. The lateral opening 422 may allow the side wall of the protrusion 420 to flex as needed. For example, a lumen 426 may extend along the axial length of the protrusion 420 and may be in communication with or connected to the lateral opening 422 of the protrusion 420. A diameter of the lumen 426 may expand to allow the adapter 400 to be positioned over the plunger 114 in a lateral direction and compressed when inserted into the lumen 112 of the barrel 102. These are just some examples.

The body 402 of the adapter 400 may include a lateral opening 428 opening toward the second lateral side 414 and aligned with the lateral opening 422 of the protrusion 420. The lateral opening 428 may define a first portion or arm 430a adjacent the top side 408 and a second arm or portion 430b adjacent the bottom side 410 which allow the body 402 of the adapter 400 to flex as needed. For example, the arms 430a, 430b may move away from one another to allow the adapter 400 to be laterally assembled with the plunger shaft 115. A lumen or aperture 432 may extend distally from the proximal side 404 of the body 402. The lumen 432 may extend co-axially with and connect to the lumen 426 of the protrusion 420 to form a continuous lumen. Further, the lumen 432 may be in communication with or connected to the lateral opening 428. In some cases, one or more slots or reliefs 434a, 434b may be radially spaced from the lumen 432 and extend partially around the perimeter thereof. The one or more reliefs 434a, 434b may extend distally from the proximal side 404 of the body 402 to the recess 416. It is contemplated that the reliefs 434a, 434b may be slits or slots configured to allow the diameter of the lumen 432 to temporarily expand to allow the adapter 400 to be positioned over the plunger 114 in a lateral direction in a similar manner to adapter 400. For example, the lateral opening 428 may have a height 452 that is less than a diameter of the plunger shaft 115.

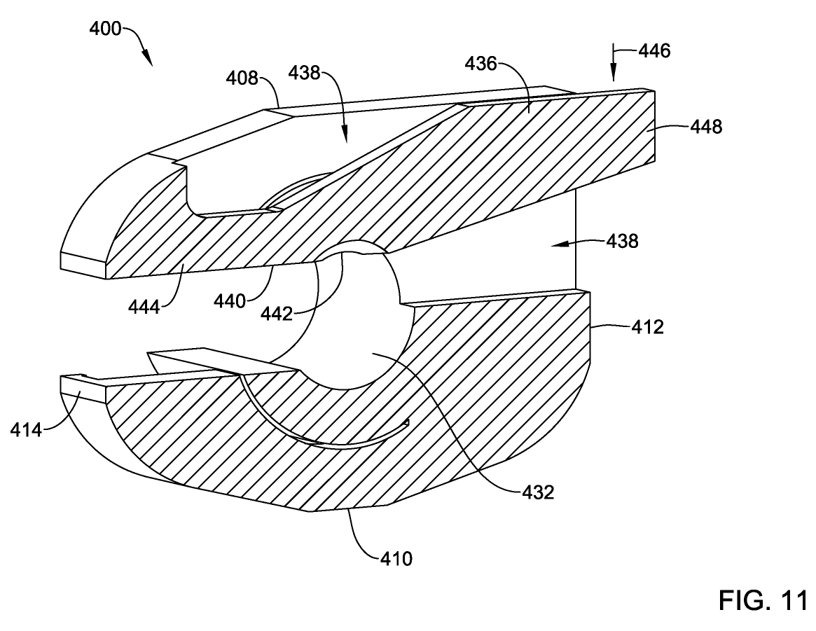

The adapter 400 may further include a compliant hinge or flexible member 436 extending along the top side 408 and laterally away from the first lateral side 412 of the body 402. Referring additionally to FIG. 11, which is a cross-sectional view of the adapter 400 taken at line 11-11 of FIG. 10B, the flexible member 436 may be movably positioned within a slot 438 formed in the body 402 of the adapter 400. The slot 438 may extend from the top side 408 of the body 402 towards the bottom side 410 thereof. In some cases, the slot 438 may extend from the top side 408 of the bottom side 410 of the body 402. Further, at least a portion of the slot 438 may extend to the first lateral side 412 of the body 402. A radially inward surface 440 of the flexible member 436 may include an internal thread 442 or more than one internal thread configured to selectively engage the external threads 126 of the plunger 114. The one or more internal threads 442 may be aligned with the lumen 432.

The flexible member 436 may be movably coupled to or formed as a deflectable section of a single monolithic structure with the body 402. For example, a region 444 of the flexible member 436 having a reduced thickness extends from the body 402. The flexible member 436 may be configured to be biased radially inwards to selectively engage the internal thread 442 with the threaded region 124 of the plunger shaft 115. In an unbiased configuration, the internal thread 442 of the flexible member 436 may be radially spaced from an outer surface of the plunger shaft 115 such that the plunger shaft 115 (e.g., between the distal end 117 of the forward stop 119 and the proximal end 123 of the plunger tip 121) is axially displaceable within the lumens 426, 432 of the adapter 400 and the lumen 112 of the barrel 102 using linear movement of the plunger 114. The user may exert a radially inwards biasing force 446 on an actuation portion 448 of the flexible member 436 to move the internal thread 442 radially inwards. The actuation portion 448 may extend laterally from the first lateral side 412 of the body 402 to allow the user to locate and actuate the flexible member 436 without visual verification. Upon exertion of the applied force 446, the flexible member 436 may bend or deflect at the reduced thickness region 444 to move radially inwards. In the absence of the applied force 446, the flexible member 436 may return to its original unbiased configuration where the internal thread 442 of the flexible member 436 may be radially spaced from an outer surface of the plunger shaft 115. The actuation portion 448 may also allow the user to move the flexible member 436 radially outwards in the event the flexible member 436 does not return to its original unbiased configurations when the radially inward force 446 is released. While not explicitly shown, the body 402 of the adapter 400 may further include an ergonomic grip pad positioned along the bottom side 410 thereof and configured to improve the gripability of the adapter 400 while the flexible member 436 is actuated. The ergonomic grip pad may be positioned adjacent to the first lateral side 412 of the body 402 opposite from the actuation portion 448. It is further contemplated that the actuation portion 448 and/or other regions of the flexible member 436 may include a plurality of grooves and ridges or other texturing to improve the gripability thereof.

When the flexible member 436 is biased radially inwards, the diameter of the lumen 432 at a proximal surface 450 of the flexible member 436 (e.g., proximal to the internal thread 442) may be less than the outermost diameter of the threaded region 124. Thus, when the plunger 114 is distally advanced with the flexible member 436 biased radially inwards, the distal end 128 of the threaded region 124 may mechanically engage the flexible member 436 proximal to the internal thread 442. The distal end 128 of the threaded region 124 may be located to allow the user to precisely inject or dispense a predetermined amount of fluid into the inflatable balloon 16 without visual verification. For example, the user may inject the fluid from the syringe system 100 into the balloon 16 without using volume lines on an outer surface of the barrel 102 to determine how much fluid has been injected into the balloon 16. Once the distal end 128 of the threaded region 124 has engaged the proximal surface 450 of the flexible member 436, the user may rotate the plunger 114 in a first direction (e.g., clockwise) to engage the distalmost thread 126 with the one or more internal threads 442. Continued rotation of the plunger 114 in the first direction may distally advance the plunger 114 within the lumen 112 of the barrel 102 to dispense additional fluid from the lumen 112 of the barrel 102 to the balloon 16 in a controlled and/or precise manner. In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102.

The user may rotate the plunger 114 until the distal end 117 of the forward stop 119 contacts the proximal side 404 of the body 402 of the adapter 400. However, the user may stop rotation of the plunger 114 at any time before the distal end 117 of the forward stop 119 contacts the proximal side 404 of the body 402 if the desired diameter of the balloon 16 has been reached. The length of the plunger shaft 115 may be selected such that when the distal end 117 of the forward stop 119 contacts the proximal side 404 of the adapter 400, the balloon 16 has been inflated to a largest size (e.g., largest diameter) or a maximum allowable volume has been dispensed. In other examples, the user may rotate the plunger 114 until the plunger seal 122 contacts a tapered distal end 188 of the lumen 112. In such an instance, the distal end 117 of the forward stop may be spaced from the proximal side 138 of the body 136 when a maximum volume of fluid has been dispensed.

It is contemplated that if the user does not wish to incrementally inflate the balloon 16 either after the minimum volume has been dispensed (to achieve the smallest balloon size) or prior to dispensing any fluid, the flexible member 436 may be released to allow the user to inflate the balloon to the largest size without requiring rotation of the plunger 114. Said differently, the syringe system 100 may be operated in a manner consistent with a typical syringe, or the flexible member 436 may be depressed to allow controlled volumes to be dispensed. It is contemplated that the adapter 400 may be used with the barrel 102 and plunger 114 in a similar manner to that described with respect to adapter 134.

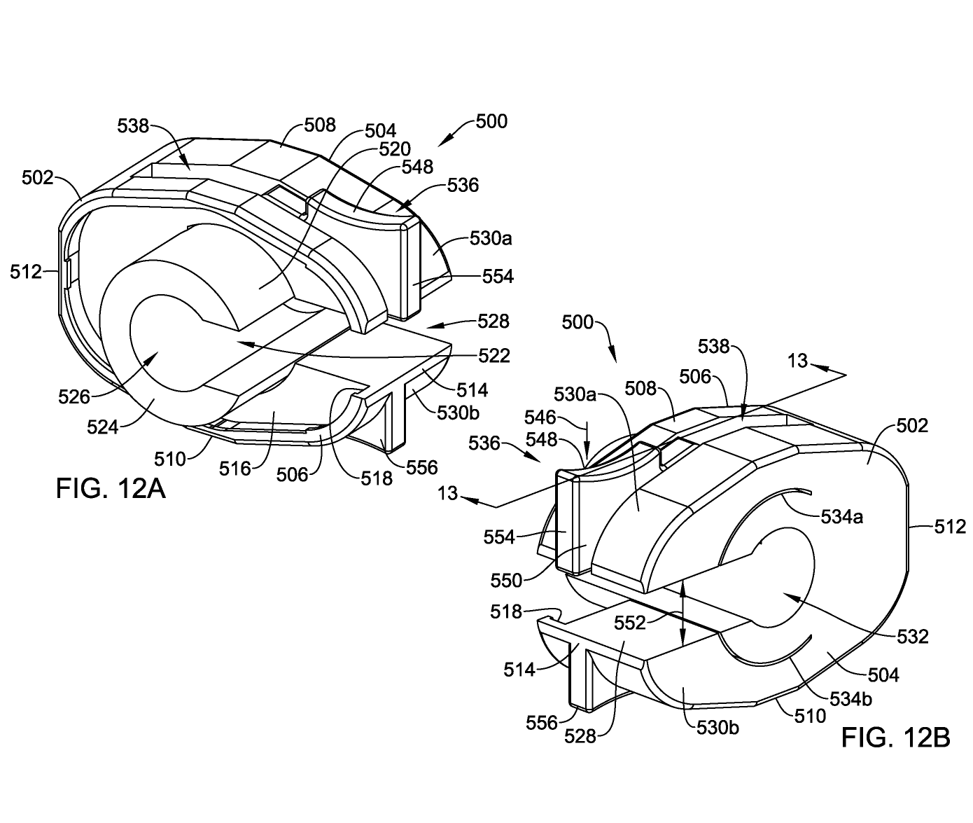

FIG. 12A is perspective view of a distal side of another illustrative adapter 500 and FIG. 12B is a perspective view of a proximal side of the adapter 500. The adapter 500 may be used with the barrel 102 and plunger 114 in place of the adapter 134 described herein. The adapter 500 may be formed as a single monolithic structure. However, this is not required. In some examples, the adapter 500 may be formed from two or more pieces or components that are subsequently coupled together. In an example configuration, the adapter 500 may be formed from one or more molded plastic components, such as, but not limited to acrylonitrile butadiene styrene (ABS). However, a variety of different manufacturing techniques and/or materials may be used to form the adapter 500.

The adapter 500 includes a housing or body 502 including a proximal side 504, a distal side 506, and at least a top side 508 and a second opposing bottom side 510. The top and bottom sides 508, 510 may each extend from or between the proximal side 504 to the distal side 506. The body 502 may further include a first lateral side 512 and an opposing second lateral side 514. The use of the terms “proximal”, “distal”, “first”, “second”, “top”, and “bottom” are not intended to limit the adapter 500 to a particular orientation, but rather facilitate discussion of relative orientation.

The distal side 506 may include a recess 516 configured to receive the barrel flange 108 therein. A groove 518 may extend at least partially around a perimeter of the recess 516. In some cases, the groove 518 may extend around an entirety of the perimeter of the recess 516. However, this is not required. The perimeter of the barrel flange 108 may form a snap fit with the groove 518 to releasably secure the adapter 500 to the barrel flange 108.

A generally C-shaped protrusion 520 may extend distally from the recess 516, and in some cases, distally beyond the distal side 506 of the body 502. In some cases, the protrusion 520 may be generally tubular with a portion of the sidewall removed along a length thereof to define a lateral opening 522. The lateral opening may be oriented towards the second lateral side 514. The protrusion 520 may be configured to be received within the lumen 112 of the barrel 102 such that a distal end 524 of the protrusion 520 is disposed within the lumen 112. For example, an outer diameter of the protrusion 520 may be less than a luminal diameter of the lumen 112. In some configurations, the outer diameter of the protrusion 520 may be approximately the same as or greater than the luminal diameter of the lumen 112 to provide a friction fit between the outer surface of the protrusion 520 and the luminal surface of the lumen 112.

The lateral opening 522 of the protrusion 520 may extend along an axial length (e.g., proximal to distal direction) thereof. The lateral opening 522 may allow the side wall of the protrusion 520 to flex as needed. For example, a lumen 526 may extend along the axial length of the protrusion 520 and may be in communication with or connected to the lateral opening 522 of the protrusion 520. A diameter of the lumen 526 may expand to allow the adapter 500 to be positioned over the plunger 114 in a lateral direction and compressed when inserted into the lumen 112 of the barrel 102. These are just some examples.

The body 502 of the adapter 500 may include a lateral opening 528 opening toward the second lateral side 514 and aligned with the lateral opening 522 of the protrusion 520. The lateral opening 528 may define a first portion or arm 530a adjacent the top side 508 and a second arm or portion 530b adjacent the bottom side 510 which allow the body 502 of the adapter 500 to flex as needed. For example, the arms 530a, 530b may move away from one another to allow the adapter 500 to be laterally assembled with the plunger shaft 115. A lumen or aperture 532 may extend distally from the proximal side 504 of the body 502. The lumen 532 may extend co-axially with and connect to the lumen 526 of the protrusion 520 to form a continuous lumen. Further, the lumen 532 may be in communication with or connected to the lateral opening 528. In some cases, one or more slots or reliefs 534a, 534b may be radially spaced from the lumen 532 and extend partially around the perimeter thereof. The one or more reliefs 534a, 534b may extend distally from the proximal side 504 of the body 502 to the recess 516. It is contemplated that the reliefs 534a, 534b may be slits or slots configured to allow the diameter of the lumen 532 to temporarily expand to allow the adapter 500 to be positioned over the plunger 114 in a lateral direction in a similar manner to adapter 500. For example, the lateral opening 528 may have a height 552 that is less than a diameter of the plunger shaft 115.

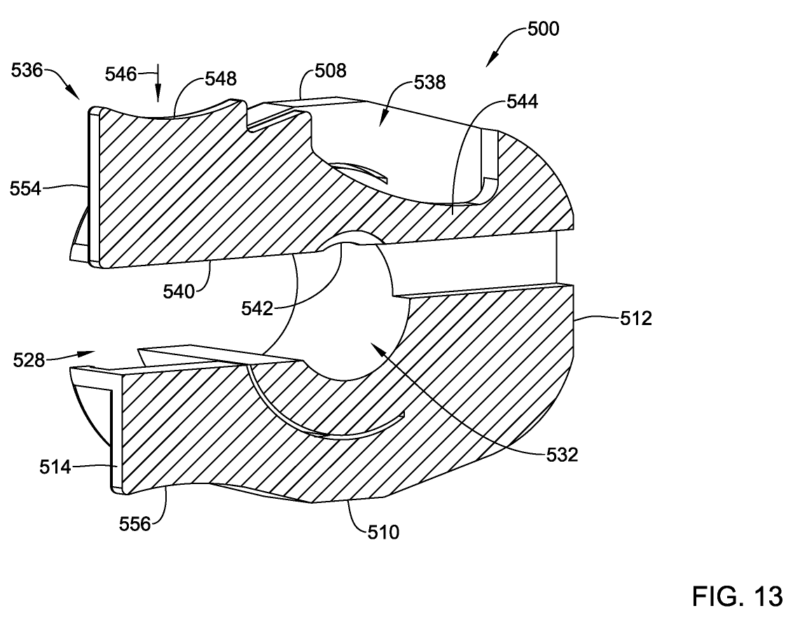

The adapter 500 may further include a compliant hinge or flexible member 536 extending along and/or extending above the top side 508 and laterally away from the second lateral side 514 of the body 502. Referring additionally to FIG. 13, which is a cross-sectional view of the adapter 500 taken at line 11-11 of FIG. 12B, the flexible member 536 may be movably positioned within a slot 538 formed in the body 502 of the adapter 500. The slot 538 may extend from the top side 508 of the body 502 towards the bottom side 510 thereof. In some cases, the slot 538 may extend from the top side 508 of the bottom side 510 of the body 502. Further, at least a portion of the slot 538 may extend to the first lateral side 512 and/or the second lateral side 514 of the body 502. A radially inward surface 540 of the flexible member 536 may include an internal thread 542 or more than one internal thread configured to selectively engage the external threads 126 of the plunger 114. The one or more internal threads 542 may be aligned with the lumen 532.

The flexible member 536 may be movably coupled to or formed as a deflectable section of a single monolithic structure with the body 502. For example, a region 544 of the flexible member 536 having a reduced thickness extends from the body 502. The flexible member 536 may be configured to be biased radially inwards to selectively engage the internal thread 542 with the threaded region 124 of the plunger shaft 115. In an unbiased configuration, the internal thread 542 of the flexible member 536 may be radially spaced from an outer surface of the plunger shaft 115 such that the plunger shaft 115 (e.g., between the distal end 117 of the forward stop 119 and the proximal end 123 of the plunger tip 121) is axially displaceable within the lumens 526, 532 of the adapter 500 and the lumen 112 of the barrel 102 using linear movement of the plunger 114. The user may exert a radially inwards biasing force 546 on an actuation portion 548 of the flexible member 536 to move the internal thread 542 radially inwards. The actuation portion 548 may extend upwards (e.g., in a direction away from the bottom side 510) from the top side 508 of the body 502 to allow the user to locate and actuate the flexible member 536 without visual verification. Upon exertion of the applied force 546, the flexible member 536 may bend or deflect at the reduced thickness region 544 to move radially inwards. In the absence of the applied force 546, the flexible member 536 may return to its original unbiased configuration where the internal thread 542 of the flexible member 536 may be radially spaced from an outer surface of the plunger shaft 115. A tab 554 may extend laterally beyond the second lateral side 514 to allow the user to move the flexible member 536 radially outwards in the event the flexible member 536 does not return to its original unbiased configurations when the radially inward force 546 is released. The body 502 of the adapter 500 may further include an ergonomic grip pad 556 positioned along the bottom side 510 thereof and configured to improve the gripability of the adapter 500 while the flexible member 536 is actuated. The ergonomic grip pad 556 may be positioned adjacent to the second lateral side 514 of the body 502 opposite from the actuation portion 548. It is further contemplated that the actuation portion 548 and/or other regions of the flexible member 536 may include a plurality of grooves and ridges or other texturing to improve the gripability thereof.

When the flexible member 536 is biased radially inwards, the diameter of the lumen 532 at a proximal surface 550 of the flexible member 536 (e.g., proximal to the internal thread 542) may be less than the outermost diameter of the threaded region 124. Thus, when the plunger 114 is distally advanced with the flexible member 536 biased radially inwards, the distal end 128 of the threaded region 124 may mechanically engage the flexible member 536 proximal to the internal thread 542. The distal end 128 of the threaded region 124 may be located to allow the user to precisely inject or dispense a predetermined amount of fluid into the inflatable balloon 16 without visual verification. For example, the user may inject the fluid from the syringe system 100 into the balloon 16 without using volume lines on an outer surface of the barrel 102 to determine how much fluid has been injected into the balloon 16. Once the distal end 128 of the threaded region 124 has engaged the proximal surface 550 of the flexible member 536, the user may rotate the plunger 114 in a first direction (e.g., clockwise) to engage the distalmost thread 126 with the one or more internal threads 542. Continued rotation of the plunger 114 in the first direction may distally advance the plunger 114 within the lumen 112 of the barrel 102 to dispense additional fluid from the lumen 112 of the barrel 102 to the balloon 16 in a controlled and/or precise manner. In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102.

The user may rotate the plunger 114 until the distal end 117 of the forward stop 119 contacts the proximal side 504 of the body 502 of the adapter 500. However, the user may stop rotation of the plunger 114 at any time before the distal end 117 of the forward stop 119 contacts the proximal side 504 of the body 502 if the desired diameter of the balloon 16 has been reached. The length of the plunger shaft 115 may be selected such that when the distal end 117 of the forward stop 119 contacts the proximal side 504 of the adapter 500, the balloon 16 has been inflated to a largest size (e.g., largest diameter) or a maximum allowable volume has been dispensed. In other examples, the user may rotate the plunger 114 until the plunger seal 122 contacts a tapered distal end 188 of the lumen 112. In such an instance, the distal end 117 of the forward stop may be spaced from the proximal side 138 of the body 136 when a maximum volume of fluid has been dispensed.

It is contemplated that if the user does not wish to incrementally inflate the balloon 16 either after the minimum volume has been dispensed (to achieve the smallest balloon size) or prior to dispensing any fluid, the flexible member 536 may be released to allow the user to inflate the balloon to the largest size without requiring rotation of the plunger 114. Said differently, the syringe system 100 may be operated in a manner consistent with a typical syringe, or the flexible member 536 may be depressed to allow controlled volumes to be dispensed. It is contemplated that the adapter 500 may be used with the barrel 102 and plunger 114 in a similar manner to that described with respect to adapter 134.

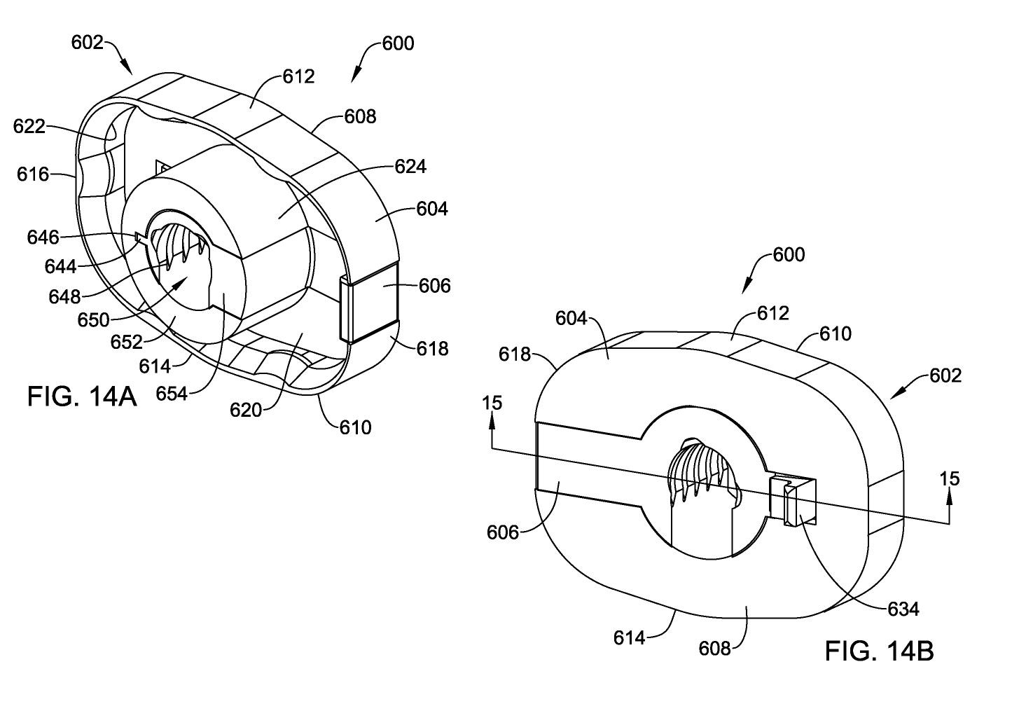

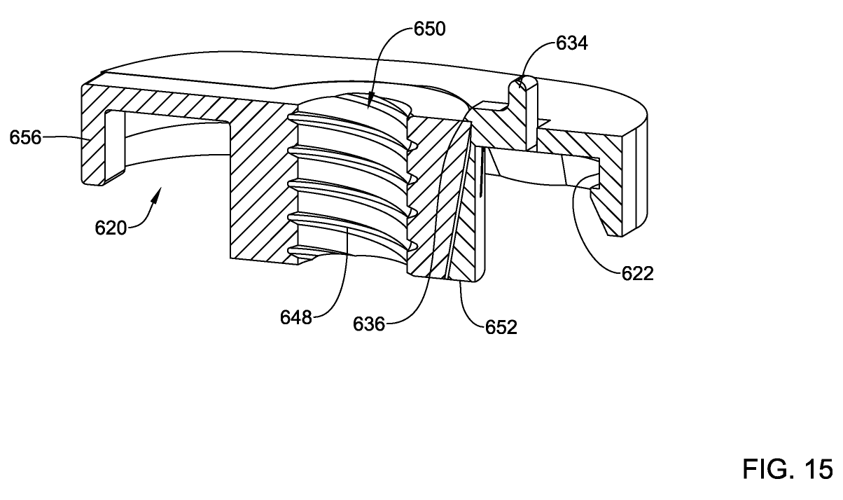

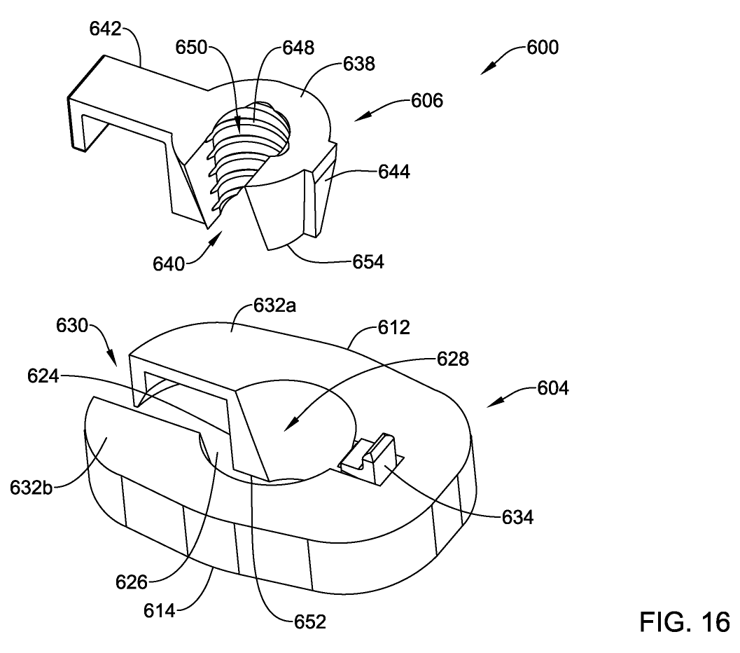

FIG. 14A is a perspective view of a distal side of another illustrative adapter 600 and FIG. 14B is a perspective view of a proximal side of the adapter 600. FIG. 15 is a cross-sectional view of the adapter 600, taken at line 15-15 of FIG. 14B. FIG. 16 is an exploded perspective view of the adapter 600. The adapter 600 may be used with the barrel 102 and plunger 114 in place of the adapter 134 described herein. Generally, the adapter 600 may allow for rapid inflation and/or deflation below the smallest balloon diameter while requiring the user to rotate the plunger 114 to inflate the balloon 16 to a diameter greater than the minimum diameter. The adapter 600 may be formed from two or more components releasably coupled together. However, this is not required. In an example configuration, the adapter 600 may be formed from one or more molded plastic components, such as, but not limited to, acrylonitrile butadiene styrene (ABS). However, a variety of different manufacturing techniques and/or materials may be used to form the adapter 600.

The adapter 600 includes a housing or body 602 including a first body member 604 and a second body member 606. The second body member 606 may be releasably coupled to the first body member 604. While the body 602 is described as having two body members 604, 606, the body 602 may include more than two body members 604, 606, as desired. The body 602 may include a proximal side 608, a distal side 610, and at least a top side 612 and a second opposing bottom side 614. The top and bottom sides 612, 614 may each extend from or between the proximal side 608 to the distal side 610. The body 602 may further include a first lateral side 616 and an opposing second lateral side 618. The use of the terms “proximal”, “distal”, “first”, “second”, “top”, and “bottom” are not intended to limit the adapter 600 to a particular orientation, but rather facilitate discussion of relative orientation.

The first body member 604 may have a generally stadium or ovular shape cross similar to the shape of the barrel flange 108 such that the barrel flange 108 may be removably coupled therewith. For example, the distal side 610 of the body 602 may include a recess 620 configured to receive the barrel flange 108 therein. In some configurations, the recess 620 may be formed in the first body member 604. A groove 622 may extend at least partially around a perimeter of the recess 620. In some cases, the groove 622 may extend around an entirety of the perimeter of the recess 620. However, this is not required. The perimeter of the barrel flange 108 may form a snap fit with the groove 622 to releasably secure the adapter 600 to the barrel flange 108.

The first body member 604 may further include a generally C-shaped protrusion 624 extending distally from the recess 620, and in some cases, distally beyond the distal side 610 of the body 602. In some cases, the protrusion 624 may be generally tubular with a portion of the sidewall removed along a length thereof to define a lateral opening 626. The lateral opening may be oriented towards the second lateral side 618. The protrusion 624 may be configured to be received within the lumen 112 of the barrel 102 such that a distal end 652 of the protrusion 624 is disposed within the lumen 112. For example, an outer diameter of the protrusion 624 may be less than a luminal diameter of the lumen 112. In some configurations, the outer diameter of the protrusion 624 may be approximately the same as or greater than the luminal diameter of the lumen 112 to provide a friction fit between the outer surface of the protrusion 624 and the luminal surface of the lumen 112.

The lateral opening 626 of the protrusion 624 may extend along an axial length (e.g., proximal to distal direction) thereof. The lateral opening 626 may allow the side wall of the protrusion 624 to flex as needed. For example, a lumen 628 may extend along the axial length of the protrusion 624 and may be in communication with or connected to the lateral opening 626 of the protrusion 624. A diameter of the lumen 628 may expand to allow the adapter 600 to be positioned over the plunger 114 in a lateral direction and compressed when inserted into the lumen 112 of the barrel 102. These are just some examples.

The first body member 604 may further include a lateral opening 630 opening toward the second lateral side 618 and aligned with the lateral opening 626 of the protrusion 624. The lateral opening 630 may define a first portion or arm 632a adjacent the top side 612 and a second arm or portion 632b adjacent the bottom side 614 which allow the first body member 604 of the adapter 600 to flex as needed. For example, the arms 632a, 632b may move away from one another to allow the first body member 604 to be laterally assembled with the plunger shaft 115.

The first body member 604 may further include an actuatable latch 634 configured to releasably secure the second body member 606 to the first body member 604. The actuatable latch 634 may be movable from a first locked configuration in which a tab or ledge 636 is disposed over a proximal surface of the second body member 606 and a second unlocked configuration (not explicitly shown) where the tab 636 is laterally displaced from the second body member 606 allowing the second body member 606 to be removed from the first body member 604 in a proximal direction. The actuatable latch 634 may be biased to the locked configuration and moved to the unlocked configuration in response to an applied lateral force (e.g., in a direction away from the second body member 606) exerted by the user.

The second body member 606 may include a distally extending generally C-shaped protrusion 638. In some cases, the protrusion 638 may be generally tubular with a portion of the sidewall removed along a length thereof to define a lateral opening 640. The lateral opening 640 may be radially offset from the lateral opening 626 in the protrusion 624 of the first body member 604. In some cases, the lateral opening 640 in the second body member 606 may be oriented towards the top or bottom sides 612, 614 of the body 602. However, this is not required. The second body member 606 may be assembled with the first body member 604 such that the protrusion 638 of the second body member 606 extends into the lumen 628 of the protrusion 624 of the first body member 604. In some cases, a distal end 654 of the protrusion 638 of the second body member 606 may align with or be adjacent to the distal end 652 of the protrusion 624 of the first body member 604.

The protrusion 638 of the second body member 606 defines a lumen 650 extending from the proximal side to the distal side thereof. One or more internal threads 648 may be formed in at least a portion of the luminal surface of the lumen 650. For example, the one or more threads 648 may not extend around an entirety of the luminal surface. However, in some cases, the one or more threads 648 may extend around an entirety of the luminal surface. The one or more threads 648 may be configured to threadingly engage the threaded region 124 of the plunger 114, as will be described in more detail herein.

The second body member 606 may further include a laterally extending arm 642. The arm 642 may extend from an outer surface of the protrusion 638 towards the second lateral side 618. The arm 642 may be sized and shaped to be positioned within the lateral opening 630 of the first body member 604 when the second body member 606 is assembled with the first body member 604. The arm 642 may have a thickness less than a length of the protrusion 638 such that the arm does not extend into the recess 620. In some cases, a side wall 656 may extend generally orthogonally from a free end of the arm 642 towards the distal side 610 of the body 602.

The second body member 606 may further include an alignment key 644. The alignment key 644 may extend from the outer surface of the protrusion 638 generally opposite from or about 180° from the arm 642. The alignment key 644 may be configured to be received within a mating slot 646 formed in a luminal surface of the protrusion 624 of the first body member 604. The key 644 and slot 646 may be circumferentially aligned with the actuatable latch 634.

In some configurations, a wall thickness of at least a portion of the protrusion 638 of the second body member 606 may taper or reduce in the distal direction. The diameter of the lumen 628 of the protrusion 624 of the first body member 604 may taper in a similar manner to create a friction fit between the first body member 604 and the second body member 606. This may limit distal advancement of the second body member 606 relative to the first body member 604.

The first and second body members 604, 606 of the adapter 600 may be assembled with the plunger 114 as separate components and subsequently coupled together. For example, the lateral opening 630 of the first body member 604 may be aligned with the plunger shaft 115 (e.g., between the forward stop 119 and the plunger tip 121). In some cases, the lateral opening 630 may be aligned with a region of the plunger shaft 115 free from threads 126. However, this is not required. The first body member 604 may then be laterally advanced over the plunger shaft 115 in a manner similar to that described with respect to FIG. 5. The lateral opening 640 of the second body member 606 may be aligned with the plunger shaft 115 (e.g., between the forward stop 119 and the plunger tip 121) proximal to the first body member 604. In some cases, the lateral opening 640 may be aligned with a region of the plunger shaft 115 free from threads 126. However, this is not required. The second body member 606 may then be laterally advanced over the plunger shaft 115 in a manner similar to that described with respect to FIG. 5. The second body member 606 may be assembled with the plunger shaft 115 in a direction generally orthogonal to the first body member 604 as the lateral opening of the 640 of the second body member 606 is circumferentially offset from the lateral opening 630 of the first body member 604. In some cases, the second body member 606 may be assembled with the plunger 114 prior to the first body member 604. Once both the first body member 604 and the second body member 606 are disposed over the plunger shaft 115, the second body member 606 may be proximally retracted (or the first body member 604 distally advanced) to position the protrusion 638 of the second body member 606 within the lumen 628 of the first body member 604. The second body member 606 may be proximally retracted until the actuatable latch 634 secures the second body member 606 relative to the first body member 604. The assembled adapter 600 may then be distally advanced to releasably couple the adapter 600 with the barrel flange 108.

The diameter or cross-sectional dimension of the lumen 650 of the second body member 606 may be greater that the diameter of the plunger shaft 115 free from threads such that the plunger 114 may be linearly displaced along a first region of the plunger shaft 115 to inflate the balloon 16 to a minimum or first predetermined volume. The distal end 128 of the threaded region 124 may be located to allow the user to precisely inject or dispense a predetermined amount of fluid into the inflatable balloon 16 without visual verification. For example, the user may inject the fluid from the syringe system 100 into the balloon 16 without using volume lines on an outer surface of the barrel 102 to determine how much fluid has been injected into the balloon 16. Once the distal end 128 of the threaded region 124 has engaged the proximal surface of the second body member 606 of the adapter 600, the user may rotate the plunger 114 in a first direction (e.g., clockwise) to engage the distalmost thread 126 with the internal threads 648. Continued rotation of the plunger 114 in the first direction may distally advance the plunger 114 within the lumen 112 of the barrel 102 to dispense additional fluid from the lumen 112 of the barrel 102 to the balloon 16 in a controlled and/or precise manner. In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102. Once the distal end 128 of the threaded region 124 is proximal to the proximal side 708 of the adapter 600, the plunger 114 may be linearly actuated to further withdraw fluid from the balloon 16.

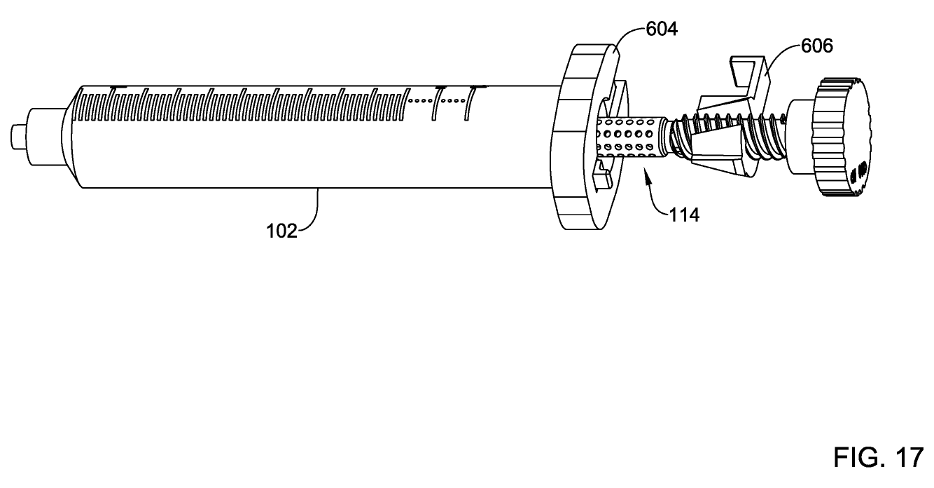

If the user requires quick deflation of the balloon 16 while the threaded region 124 of the plunger 114 is engaged with the internal threads 648 of the second body member 606, the user may actuate the actuatable latch 634 while proximally retracting the plunger 114. This may allow the second body member 606 to separate from the first body member 604, as shown in FIG. 17 which is a perspective view of the disassembled adapter 600 coupled to the barrel 102 and plunger 114, to allow for rapid removal of fluid from the balloon 16. Said differently, fluid can be removed from the balloon 16 without requiring rotation of the plunger 114.

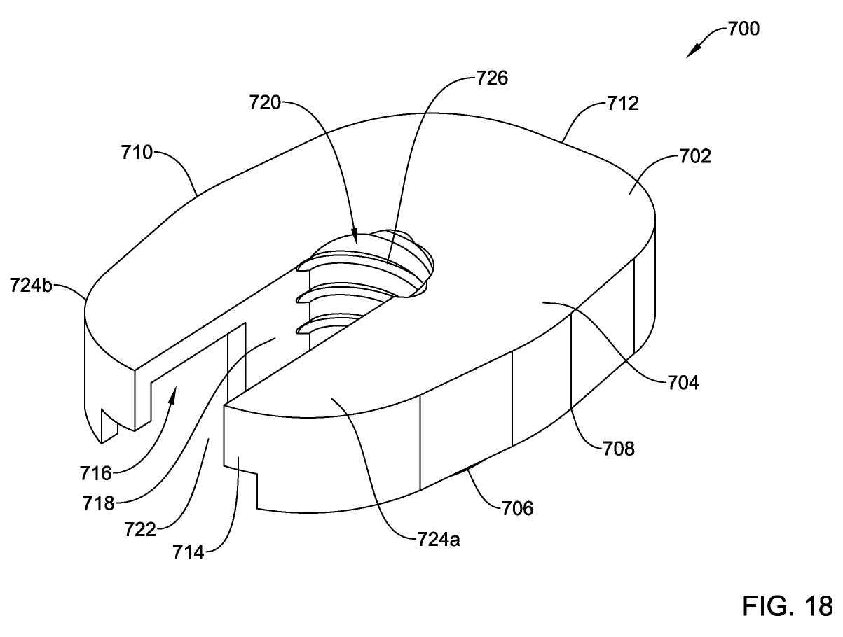

FIG. 18 is a proximal perspective view of another illustrative adapter 700 that may be used with the barrel 102 and plunger 114 in place of the adapter 134 described herein. Generally, the adapter 700 may allow for rapid inflation and/or deflation below the smallest balloon diameter while requiring the user to rotate the plunger 114 to inflate the balloon 16 to a diameter greater than the minimum diameter. In an example configuration, the adapter 700 may be formed from one or more molded plastic components, such as, but not limited to, acrylonitrile butadiene styrene (ABS). However, a variety of different manufacturing techniques and/or materials may be used to form the adapter 700.

The adapter 700 includes a housing or body 702. The body 702 may include a proximal side 704, a distal side 706, and at least a top side 708 and a second opposing bottom side 710. The top and bottom sides 708, 710 may each extend from or between the proximal side 704 to the distal side 706. The body 702 may further include a first lateral side 712 and an opposing second lateral side 714. The use of the terms “proximal”, “distal”, “first”, “second”, “top”, and “bottom” are not intended to limit the adapter 700 to a particular orientation, but rather facilitate discussion of relative orientation.

The body 702 may have a generally stadium or ovular shape cross similar to the shape of the barrel flange 108 such that the barrel flange 108 may be removably coupled therewith. For example, the distal side 706 of the body 702 may include a recess 716 configured to receive the barrel flange 108 therein. A groove (not explicitly shown) may extend at least partially around a perimeter of the recess 716. In some cases, the groove may extend around an entirety of the perimeter of the recess 716. However, this is not required. The perimeter of the barrel flange 108 may form a snap fit with the groove to releasably secure the adapter 700 to the barrel flange 108.

The body 702 may further include a generally C-shaped protrusion 718 extending distally from the recess 716, and in some cases, distally beyond the distal side 706 of the body 702. In some cases, the protrusion 718 may be generally tubular with a portion of the sidewall removed along a length thereof to define a lateral opening. The lateral opening may be oriented towards the second lateral side 714. The protrusion 718 may be configured to be received within the lumen 112 of the barrel 102 such that a distal end of the protrusion 718 is disposed within the lumen 112. For example, an outer diameter of the protrusion 718 may be less than a luminal diameter of the lumen 112. In some configurations, the outer diameter of the protrusion 718 may be approximately the same as or greater than the luminal diameter of the lumen 112 to provide a friction fit between the outer surface of the protrusion 718 and the luminal surface of the lumen 112.

The lateral opening of the protrusion 718 may extend along an axial length (e.g., proximal to distal direction) thereof. The lateral opening may allow the side wall of the protrusion 718 to flex as needed. For example, a lumen 720 may extend along the axial length of the protrusion 718 and may be in communication with or connected to the lateral opening of the protrusion 718. A diameter of the lumen 720 may expand to allow the adapter 700 to be positioned over the plunger 114 in a lateral direction and compressed when inserted into the lumen 112 of the barrel 102. These are just some examples.

The body 702 may further include a lateral opening 722 opening toward the second lateral side 714 and aligned with the lateral opening of the protrusion 718. The lateral opening 722 may define a first portion or arm 724a adjacent the top side 708 and a second arm or portion 724b adjacent the bottom side 710 which allow the body 702 of the adapter 700 to flex as needed. For example, the arms 724a, 724b may move away from one another to allow the body 702 to be laterally assembled with the plunger shaft 115.

One or more internal threads 726 may be formed in at least a portion of the luminal surface of the lumen 720. For example, the one or more threads 726 may not extend around an entirety of the luminal surface. However, in some cases, the one or more threads 726 may extend around an entirety of the luminal surface. The one or more threads 726 may be configured to threadingly engage the threaded region 124 of the plunger 114, as will be described in more detail herein.

To assemble the adapter 700 with the plunger 114, the lateral opening 722 of the body 702 may be aligned with the plunger shaft 115 (e.g., between the forward stop 119 and the plunger tip 121). In some cases, the lateral opening 722 may be aligned with a region of the plunger shaft 115 free from threads 126. However, this is not required. The body 702 may then be laterally advanced over the plunger shaft 115 in a manner similar to that described with respect to FIG. 5. The adapter 700 may then be distally advanced to releasably couple the adapter 700 with the barrel flange 108.

The diameter or cross-sectional dimension of the lumen 720 of the body 702 may be greater that the diameter of the plunger shaft 115 free from threads such that the plunger 114 may be linearly displaced along a first region of the plunger shaft 115 to inflate the balloon 16 to a minimum or first predetermined volume. The distal end 128 of the threaded region 124 may be located to allow the user to precisely inject or dispense a predetermined amount of fluid into the inflatable balloon 16 without visual verification. For example, the user may inject the fluid from the syringe system 100 into the balloon 16 without using volume lines on an outer surface of the barrel 102 to determine how much fluid has been injected into the balloon 16. Once the distal end 128 of the threaded region 124 has engaged the proximal surface of the body 702 of the adapter 700, the user may rotate the plunger 114 in a first direction (e.g., clockwise) to engage the distalmost thread 126 with the internal threads 726. Continued rotation of the plunger 114 in the first direction may distally advance the plunger 114 within the lumen 112 of the barrel 102 to dispense additional fluid from the lumen 112 of the barrel 102 to the balloon 16 in a controlled and/or precise manner. In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102. Once the distal end 128 of the threaded region 124 is proximal to the proximal side 704 of the adapter 700, the plunger 114 may be linearly actuated to further withdraw fluid from the balloon 16.

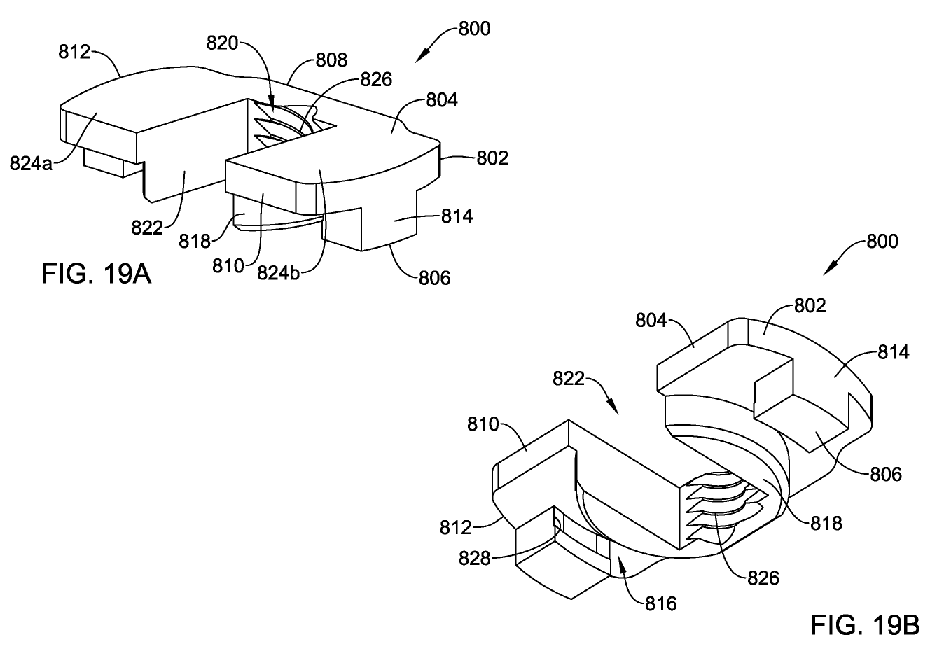

FIG. 19A is perspective view of a proximal side of another illustrative adapter 800 and FIG. 19B is a perspective view of a proximal side of the adapter 800. The adapter 800 may be used with the barrel 102 and plunger 114 in place of the adapter 134 described herein. Generally, the adapter 800 position the internal threads off-axis from the primary axis of the syringe operation. The user can choose to engage the smaller diameter portion which contains the threads for precise control or operate the syringe in a traditional manner with linear actuation of the plunger 114. In an example configuration, the adapter 800 may be formed from one or more molded plastic components, such as, but not limited to, acrylonitrile butadiene styrene (ABS). However, a variety of different manufacturing techniques and/or materials may be used to form the adapter 800.

The adapter 800 includes a housing or body 802. The body 802 may include a proximal side 804, a distal side 806, and at least a top side 808 and a second opposing bottom side 810. The top and bottom sides 808, 810 may each extend from or between the proximal side 804 to the distal side 806. The body 802 may further include a first lateral side 812 and an opposing second lateral side 814. The use of the terms “proximal”, “distal”, “first”, “second”, “top”, and “bottom” are not intended to limit the adapter 800 to a particular orientation, but rather facilitate discussion of relative orientation.

The body 802 may have a generally stadium or ovular shape cross similar to the shape of the barrel flange 108 such that the barrel flange 108 may be removably coupled therewith. For example, the distal side 806 of the body 802 may include a recess 816 configured to receive the barrel flange 108 therein. A groove 828 may extend at least partially around a perimeter of the recess 816. In some cases, the groove 828 may extend around an entirety of the perimeter of the recess 816. However, this is not required. The perimeter of the barrel flange 108 may form a snap fit with the groove 828 to releasably secure the adapter 800 to the barrel flange 108.

The body 802 may further include a generally C-shaped protrusion 818 extending distally from the recess 816, and in some cases, distally beyond the distal side 806 of the body 802. In some cases, the protrusion 818 may be generally tubular with a portion of the sidewall removed along a length thereof to define a lateral slot or opening 822. The lateral opening 822 may be oriented towards the bottom side 810. However, this is not required. The opening 822 may be oriented in other directions, as desired. The protrusion 818 may be configured to be received within the lumen 112 of the barrel 102 such that a distal end of the protrusion 818 is disposed within the lumen 112. For example, an outer diameter of the protrusion 818 may be less than a luminal diameter of the lumen 112. In some configurations, the outer diameter of the protrusion 818 may be approximately the same as or greater than the luminal diameter of the lumen 112 to provide a friction fit between the outer surface of the protrusion 818 and the luminal surface of the lumen 112.

The lateral opening 822 of the protrusion 818 may extend along an axial length (e.g., proximal to distal direction) thereof and to the proximal side 804 of the body 802. The lateral opening 822 may allow the side wall of the protrusion 818 and the body 802 to flex as needed. For example, a lumen 820 may extend along the axial length of the protrusion 818 and may be in communication with or connected to the lateral opening 822 of the protrusion 818. A diameter of the lumen 820 may expand to allow the adapter 800 to be positioned over the plunger 114 in a lateral direction and compressed when inserted into the lumen 112 of the barrel 102. These are just some examples.

The lateral opening 822 may define a first portion or arm 824a adjacent the first lateral side 812 and a second arm or portion 824b adjacent the second lateral side 814 which allow the body 802 of the adapter 800 to flex as needed. For example, the arms 824a, 824b may move away from one another to allow the body 802 to be laterally assembled with the plunger shaft 115.

One or more internal threads 826 may be formed in at least a portion of the luminal surface of the lumen 820. For example, the one or more threads 826 may not extend around an entirety of the luminal surface. The one or more threads 826 may be configured to threadingly engage the threaded region 124 of the plunger 114, as will be described in more detail herein. In some configurations, the one or more threads 826 may be radially offset from an outer surface of the plunger shaft 115 when the adapter 800 is assembled with the barrel 102 and plunger 114. For example, to engage the threaded region 124 of the plunger shaft 115 with the internal threads 826, the user can push or bias the plunger 114 off-axis and into the internal threads 826 on the adapter to allow fine control of the balloon size.

To assemble the adapter 800 with the plunger 114, the lateral opening 822 of the body 802 may be aligned with the plunger shaft 115 (e.g., between the forward stop 119 and the plunger tip 121). In some cases, the lateral opening 822 may be aligned with a region of the plunger shaft 115 free from threads 126. However, this is not required. The body 802 may then be laterally advanced over the plunger shaft 115 in a manner similar to that described with respect to FIG. 5. The adapter 800 may then be distally advanced to releasably couple the adapter 800 with the barrel flange 108.

When the plunger 114 is on axis with a longitudinal axis of the barrel 102, the diameter or cross-sectional dimension of the lateral opening 822 and/or lumen 820 of the body 802 may be greater that the diameter of the plunger shaft 115 such that the plunger 114 may be linearly displaced along an entirety of the plunger shaft 115 to inflate the balloon 16 any desired volume with minor friction between an outer surface of the plunger shaft 115 and the wall of the adapter 800. To enable precise control of the inflation volume and/or diameter, the user can push or bias the plunger 114 off-axis and into the internal threads 826 on the adapter 800. The user may then rotate the plunger 114 in a first direction to inflate the balloon 16 and a second opposing direction to deflate the balloon 16. At any time, the biasing force on the plunger 114 may be removed to allow the plunger 114 to be linearly actuated to quickly inflate or deflate the balloon 16.

In some cases, the threads 126 may be a standard thread. In other configurations, the threads 126 may be a custom configuration such that a 360º rotation (or a single rotation) of the plunger 114 corresponds to a predetermined volume of dispensed fluid. For example, a single rotation of the plunger 114 may correspond to axial movement of the plunger 114 which dispenses 0.25 milliliters of fluid. This is just one example. Alternatively, or additionally, a single rotation of the plunger 114 may correspond to a predetermined change in balloon diameter. For example, a single rotation of the plunger 114 may correspond to axial movement of the plunger 114 which dispenses enough fluid to expand the balloon 16 by 0.25 millimeters. Rotating the plunger 114 in a second direction, opposite the first direction, may proximally retract to the plunger 114 to draw fluid back into lumen 112 of the barrel 102. Once the distal end 128 of the threaded region 124 is proximal to the proximal side 804 of the adapter 800, the plunger 114 may be linearly actuated to further withdraw fluid from the balloon 16.

The materials that can be used for the various components of the systems presently disclosed may include those commonly associated with medical devices. For simplicity purposes, the following discussion makes reference to system 100 referenced above. However, this is not intended to limit the devices and methods described herein, as the discussion may be applied to other similar devices and/or components of devices disclosed herein.

The system 100 and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium- molybdenum alloys (e.g., UNS: R30035 such as MP35-N® and the like), nickel- molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A syringe system for controlled inflation, comprising:
a barrel extending from a proximal end to a distal end and including a barrel flange;
a plunger movably disposed within the barrel and extending from a proximal end to a distal end, the plunger including a plunger flange and a threaded region;
an adapter releasably coupled to the barrel flange, the adapter including:
a body having a proximal side and a distal side;
a flexible member extending from the body; and
an internal thread on the flexible member configured to selectively engage the threaded region of the plunger.

2. The syringe system of claim 1, wherein the flexible member is movable between:
a first configuration where the internal thread is radially spaced from the threaded region of the plunger; and
a second configuration where the internal thread engages the threaded region of the plunger.

3. The syringe system of claim 2, wherein the flexible member includes an actuation portion configured to move the flexible member between the first and second configurations.

4. The syringe system of claim 1, wherein the adapter includes a protrusion extending distally from the distal side of the body, the protrusion configured to be received within a lumen of the barrel.

5. The syringe system of claim 4, wherein the protrusion includes a lateral opening.

6. The syringe system of claim 1, wherein the threaded region includes at least one of:
a single helix thread;
a double start thread; or
a triple start thread.

7. The syringe system of claim 1, wherein the plunger includes a forward stop extending distally from the plunger flange.

8. The syringe system of claim 1, wherein the plunger includes a plurality of indentations extending along a length thereof.

9. The syringe system of claim 1, wherein the plunger flange includes visual indicia indicating rotational direction.

10. The syringe system of claim 1, wherein the adapter includes an ergonomic grip pad.

11. A syringe adapter system, comprising:
a first body member including a recess configured to receive a barrel flange of a syringe barrel;
a second body member releasably coupled to the first body member;
wherein at least one of the first and second body members includes an internal thread configured to engage a threaded region of a plunger.

12. The syringe adapter system of claim 11, wherein the first body member includes an actuatable latch configured to releasably secure the second body member to the first body member.

13. The syringe adapter system of claim 11, wherein the second body member includes an alignment key configured to be received within a mating slot of the first body member.

14. The syringe adapter system of claim 11, wherein the first body member includes a protrusion configured to be received within a lumen of the syringe barrel.

15. The syringe adapter system of claim 14, wherein the second body member includes a protrusion configured to be received within a lumen of the protrusion of the first body member.

16. A method of using a syringe system, comprising:
assembling an adapter with a barrel and plunger;
filling the barrel with inflation fluid;
coupling the syringe to a balloon catheter;
actuating a flexible member of the adapter;
distally advancing the plunger to dispense fluid to inflate a balloon to a first diameter.

17. The method of claim 16, further comprising rotating the plunger to engage a threaded region of the plunger with an internal thread of the flexible member.

18. The method of claim 17, further comprising continuing to rotate the plunger to incrementally increase the diameter of the balloon.

19. The method of claim 16, further comprising releasing the flexible member to allow linear actuation of the plunger.

20. The method of claim 16, wherein assembling the adapter includes:
laterally advancing the adapter over the plunger; and
distally advancing the adapter to engage a barrel flange.
